(19) United States
(12) Patent Application Publication
(10) Pub. No.: US 2025/0210199 A1
(43) Pub. Date: Jun. 26, 2025

(54) SYSTEMS AND METHODS FOR PREDICTING COSMETIC DERMATOLOGY TREATMENT PLANS AND HUMAN SKIN CHARACTERISTICS WITH ARTIFICIAL INTELLIGENCE

(71) Applicant: Katarina Kesty, St. Petersburg, FL (US)

(72) Inventor: Katarina Kesty, St. Petersburg, FL (US)

(21) Appl. No.: 19/077,640

(22) Filed: Mar. 12, 2025

Related U.S. Application Data
(63) Continuation-in-part of application No. 18/904,489, filed on Oct. 2, 2024.
(60) Provisional application No. 63/542,142, filed on Oct. 3, 2023.

(51) Int. Cl. G16H 50/20; G06T 7/00; G16H 10/20; G16H 10/60; G16H 20/40

(52) U.S. Cl. CPC G16H 50/20; G06T 7/0012; G16H 10/20; G16H 10/60; G16H 20/40; G06T 2207/20081; G06T 2207/30088; G06T 2207/30201

(57) ABSTRACT

Systems and methods for predicting cosmetic dermatology treatment plans and human skin characteristics with artificial intelligence are disclosed. According to an aspect, a system comprises one or more processors and memory comprising an expert inference engine. The expert inference engine is configured to receive data that indicates skin characteristics of a patient. Further, the expert inference engine is configured to maintain an expert system knowledge base for operating a therapeutic laser. The expert inference engine is also configured to determine settings for the therapeutic laser based on the expert system knowledge base and the skin characteristics of the patient. Further, the expert inference engine is configured to a user interface configured to present the determined settings for treating the patient.

### CROSS-REFERENCE TO RELATED APPLICATION

This application is a continuation-in-part of U.S. Non-Provisional patent application Ser. No. 18/904,489, filed on Oct. 2, 2024, which in turn claims priority to U.S. Provisional Patent Application No. 63/542,142, filed on Oct. 3, 2023, all incorporated herein by reference.

### TECHNICAL FIELD

The presently disclosed subject matter relates generally to artificial intelligence and dermatology. Particularly, the presently disclosed subject matter relates to use of artificial intelligence for prediction of therapeutic laser settings required for treatment of diverse dermatologic conditions, prediction of human skin characteristics from photographs, and prediction of wrinkle treatment plans including wrinkle treatments involving injected medication.

### BACKGROUND

In cosmetic dermatology, lasers and injected medications are powerful treatments that can help resolve a variety of skin conditions. Lasers are defined as Light Amplification by Stimulated Emission of Radiation. Lasers are used in dermatology to treat a wide variety of skin diseases and conditions, including rosacea, port-wine stains, hemangiomas, cafe au lait macules, telangiectasias, wrinkles, warts, scars, skin redness, Kaposi sarcoma, poikiloderma of Civatte, freckles, birthmarks, nevi, and sun damage. Lasers can also be used to remove unwanted hair and tattoos. Furthermore, lasers can be used to treat precancerous lesions (actinic keratoses) and prevent skin cancers. Numerous types of lasers along the electromagnetic spectrum can be used in dermatology including CW, quasi-CW, long-pulsed, short-pulsed, and picosecond lasers. There are many wavelengths of lasers used commonly in cosmetic dermatology including 532 nanometers (nm), 595 nm, 755 nm, 1064 nm, 1460 nm, 1550 nm, 1927 nm, 2940 nm, and 10,600 nm. Each wavelength targets a specific part of the skin (also known as the “chromophore”) and if set at the correct configuration for the patient, can treat various skin disorders and the signs of the aging process and sun damage. According to the American Society for Dermatologic Surgery Consumer Survey, in 2013 30% of consumers were considering getting a laser procedure; in 2023 this has skyrocketed to 70% of consumers that were interested in getting laser to improve their appearance. In order to achieve the desired therapeutic outcome, a laser must be configured in the proper way depending upon the patient's characteristics, including the skin condition to be treated, the anatomical location of the skin condition, and the patient's age. There are numerous laser devices and each laser comes with its own configuration options. Furthermore, within the various wavelengths that are studied for dermatologic conditions, there are multiple brands of lasers that have the same wavelength. Each brand of lasers has unique configuration options for the physician that need to be learned and/or mastered before any patients can be treated. This makes laser training very in depth and complex as each new laser device has unique settings. For example, if one wanted to treat a cherry angioma on the face one would want a laser with a 532 wavelength. There are more than 25 different lasers and laser brands on the market that have a 532 wavelength laser. Determining the proper laser and laser settings to use requires advanced training. However, in spite of high interest in laser treatment from both medical professionals and patients, the advanced training in use of lasers is not widely available. In 2023, approximately 530 people graduated from Dermatology residencies. Only 24 spots were available for cosmetic and laser fellowships through the American Society for Dermatologic Surgery. There is a higher demand for Lasers in Dermatology than there are physicians and non-physician providers who are skilled and experienced in lasers. This results in fewer patients receiving laser treatments for dermatologic conditions as well as a high complication rate from untrained and/or inexperienced providers doing laser procedures incorrectly or ineffectively. For example, there are a highly limited number of spots in cosmetic dermatology fellowships where someone who graduated from a dermatology residency program could learn the proper use of lasers.

A challenge facing dermatology is under-representation of darker skin types in literature, case series, and studies of the skin and skin diseases. The Fitzpatrick skin type scale ranges from 1 to 6 with 1, 2, and 3 being lighter skin types and 4, 5, and 6 being darker skin types. This scale is useful because it allows practitioners to classify skin types and study how dermatologic conditions and diseases show up in various skin tones. At present, there is no standardization of the classification of Fitzpatrick skin type other than general categories. For example, Fitzpatrick skin Type 1 is described as “always burns, never tans”. The challenge is that different observers may classify patients into different Fitzpatrick skin types. There is a similar challenge in the rating scales of the Glogau Wrinkle Scale, the Fitzpatrick Wrinkle Scale, the Kesty pigmentation scale, and the Kesty redness scale. When examining a patient or designing a study, a standardized classification for each of these categories would be beneficial to practitioners and scientists conducting studies.

In view of the foregoing, there is an ongoing need for improved systems and techniques for treatment of diverse dermatologic conditions, prediction of human skin characteristics, and generation of wrinkle treatment plans.

### SUMMARY

An objective of the presently disclosed subject matter is to make laser therapy more accessible via creation of an artificial intelligence (AI) system that captures the expertise needed to safely and effectively use lasers. The AI system described includes an expert system that takes patient characteristics as input and predicts the appropriate laser settings as output. This AI system may additionally include a machine learning component in the form of a computer vision model that predicts skin features from one or more patient photographs. The predicted skin features may subsequently be used as inputs to the expert system. The predicted skin features may also be used to standardize results for future laser research. A purpose of the machine learning/computer vision model is to capture the expertise needed to predict skin features from patient appearance, which is a skill that not all clinicians may possess. Skin features predicted may include, but are not limited to, measures of color, propensity to burn, redness, pigmentation, and/or wrinkle severity. Thus, incorporation of the machine learning/computer vision model may expand the availability of the expert system technology to a wider user base.

Another objective of the presently disclosed subject matter is to provide an AI expert system that produces a treatment plan for the treatment of the aging face including wrinkles. As the skin loses collagen, wrinkles caused by muscle movement that were once “dynamic” or temporary, become more prominent and “static”. There are several injectable neuromodulating medications (herein referred to as “neurotoxins”), including Botox®, Dysport®, Jeaveau®, Xeomin®, and Daxxify®, that are currently Food and Drug Administration-(FDA) approved to soften the appearance of static wrinkles. These medications work by inhibiting the release of acetylcholine from the presynaptic motor neurons, which blocks the chemical signal that causes muscle contraction. By inhibiting muscle contraction, the wrinkle is not able to be formed, thus relaxing the wrinkle. The products that are FDA-approved to treat wrinkles by neurotoxin are similar in their mechanism of action and clinical effect, but they vary in their injection pattern, dosing, and chemical structure. Having different dosing and injection patterns for each neurotoxin product poses a challenge to physicians and non-physician providers who wish to provide neurotoxin services to their patients. Furthermore, similar to lasers, the demand for these injectable neurotoxins is greater than the available treatment capacity of physicians and non-physician providers who currently are trained at injecting these substances. The AI system described in this application may enable more patients to receive wrinkle relaxation services, as the AI system provides a treatment plan for the physician or non-physician provider to inject the wrinkle relaxers in the correct locations and doses to provide the optimal cosmetic outcome for the patient.

The presently disclosed subject matter relates to systems and methods for predicting cosmetic dermatology treatment plans and human skin characteristics using AI. According to an aspect, a computer program product is provided. The computer program product comprises a computer readable storage medium having program instructions embodied therewith. The program instructions are executable by a computing device to cause the computing device to receive input from a user specifying patient characteristics including the skin condition needing treatment. Further, the program instructions are executable by a computing device to cause the computing device to apply an expert system inference engine to an expert system knowledge base to predict a cosmetic dermatology treatment plan based on the user-supplied patient characteristics. The program instructions are also executable by a computing device to cause the computing device to display the predicted cosmetic dermatology treatment plan to the user through a user interface.

According to another aspect, a system comprises one or more processors and memory comprising an expert inference engine. The expert inference engine is configured to receive data that indicates skin characteristics of a patient. Further, the expert inference engine is configured to maintain an expert system knowledge base for operating a therapeutic laser. The expert inference engine is also configured to determine settings for the therapeutic laser based on the expert system knowledge base and the skin characteristics of the patient. Further, the expert inference engine is configured to a user interface configured to present the determined settings for treating the patient.

According to another aspect, the subject matter described in this application is an AI system that captures cosmetic dermatology expertise to predict the proper settings or doses needed to safely administer laser treatment or injected medications to treat cosmetic dermatology concerns.

### BRIEF DESCRIPTION OF THE DRAWINGS

Having thus described the presently disclosed subject matter in general terms, reference will now be made to the accompanying Drawings, which are not necessarily drawn to scale, and listed hereinbelow.

FIG. 1A is a block diagram of a system operable to construct an expert system that includes a knowledge base and inference engine and runs on a user's computing device to present expert system laser settings output to a user in accordance with embodiments of the present disclosure;

FIG. 1B is a block diagram of a system operable to construct an expert system and a machine learning system that run on a user's computing device to present expert system laser settings output to a user in accordance with embodiments of the present disclosure;

FIG. 1C is a block diagram of a system for constructing an expert system that includes a knowledge base and inference engine and runs on a cloud computing device to present expert system cosmetic dermatology treatment plan output to a user in accordance with embodiments of the present disclosure;

FIG. 1D is a block diagram of a system for constructing an expert system and a machine learning system that run on a cloud computing device to present expert system cosmetic dermatology treatment plan output to a user in accordance with embodiments of the present disclosure;

FIG. 2A is a flowchart of an example method in accordance with embodiments of the present disclosure;

FIG. 2B is a flowchart of a method for constructing an expert system that includes a knowledge base and inference engine and for presenting the expert system cosmetic dermatology treatment plan output to a user in accordance with embodiments of the present disclosure;

FIG. 2C is a flowchart of a method for constructing an expert system and a machine learning system that together enable presenting expert system cosmetic dermatology treatment plan output to a user in accordance with embodiments of the present disclosure;

FIG. 2D is a flowchart of another method for constructing an expert system and a machine learning system that together enable presenting expert system cosmetic dermatology treatment plan output to a user in accordance with embodiments of the present disclosure;

FIG. 3A is an example of the user interface for the Cutera® Excel® V laser. This is a 532 nm wavelength used to treat telangiectasias and other vascular lesions. The wavelength can be changed from 532 nm to 1064 nm depending on the skin condition being treated. Included configuration options include fluence, pulse duration, wavelength, spot size, cooling and repetition rate;

FIG. 3B is an example of the Cutera® Excel® V laser. The device features two handpieces and a laser settings screen. Interface for this laser is displayed in FIG. 3A;

FIG. 3C is an example of a user interface for the Sciton® Joule® laser. This is the 2940 wavelength laser used to treat sun damage, pre-cancers, wrinkles, and other dermatologic conditions. Depth, percentage of area treated, spot size and coagulation can be changed by the user;

FIG. 3D is the Sciton® Joule® laser. The interface for this laser is displayed in FIG. 3C;

FIG. 4 is an example of potential injection point diagrams with dosing information for wrinkle relaxation;

FIG. 5 is an example computing architecture for an AI cloud platform for cosmetic dermatology, in one or more embodiments;

FIG. 6 illustrates an example server-side cosmetic dermatology system, in one or more embodiments;

FIG. 7 illustrates an example ecosystem, in one or more embodiments;

FIG. 8 is a flowchart of an example process for utilizing an AI cloud platform for cosmetic dermatology (e.g., the server-side cosmetic dermatology system), in one or more embodiments;

FIG. 9 is a flowchart of another example process for utilizing an AI cloud platform for cosmetic dermatology (e.g., the server-side cosmetic dermatology system), in one or more embodiments;

FIG. 10 is a flowchart of an example process for training machine learning models, in one or more embodiments;

FIG. 11 is a flowchart of an example process implemented by an AI cloud platform (e.g., the server-side cosmetic dermatology system), in one or more embodiments; and

FIG. 12 is a high-level block diagram showing an information processing system comprising a computer system useful for implementing the disclosed embodiments.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

The functional units described in this specification have been labeled as computing devices. A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a retail environment and communicatively connected to other computing devices (e.g., point of sale (POS) equipment or computers) for managing accounting, purchase transactions, and other processes within the retail environment. In another example, a computing device may be a mobile computing device such as, for example, but not limited to, a smart phone, a cell phone, a pager, a personal digital assistant (PDA), a mobile computer with a smart phone client, or the like. In another example, a computing device may be any type of wearable computer, such as a computer with a head-mounted display (HMD), or a smart watch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONE® smart phone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smart phones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, REFLEX, iDEN, TETRA, DECT, DataTAC, Mobitex, EDGE and other 2G, 3G, 4G, 5G, and LTE technologies, and it operates with many handheld device operating systems, such as EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone or smart watch that operates over GPRS (General Packet Radio Services), which is a data technology for GSM networks or operates over Near Field Communication, e.g., Bluetooth®. In addition to a conventional voice communication, a given mobile device can communicate with another such device via many different types of message transfer techniques, including Bluetooth®, Near Field Communication, SMS (short message service), enhanced SMS (EMS), multi-media message (MMS), email WAP, paging, or other known or later-developed wireless data formats. Although many of the examples provided herein are implemented on smart phones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single dataset, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

As used herein, the term “memory” is generally a storage device of a computing device. Examples include, but are not limited to, read-only memory (ROM) and random access memory (RAM).

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

As referred to herein, the terms “computing device” and “entities” should be broadly construed and should be understood to be interchangeable. They may include any type of computing device, for example, a server, a desktop computer, a laptop computer, a smart phone, a cell phone, a pager, a personal digital assistant (PDA, e.g., with GPRS NIC), a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The display object can be displayed on a display screen of a mobile device and can be selected by and interacted with by a user using the interface. In an example, the display of the mobile device can be a touch screen, which can display the display icon. The user can depress the area of the display screen at which the display icon is displayed for selecting the display icon. In another example, the user can use any other suitable interface of a mobile device, such as a keypad, to select the display icon or display object. For example, the user can use a track ball or times program instructions thereon for causing a processor to carry out aspects of the present disclosure.

As referred to herein, a computer network may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

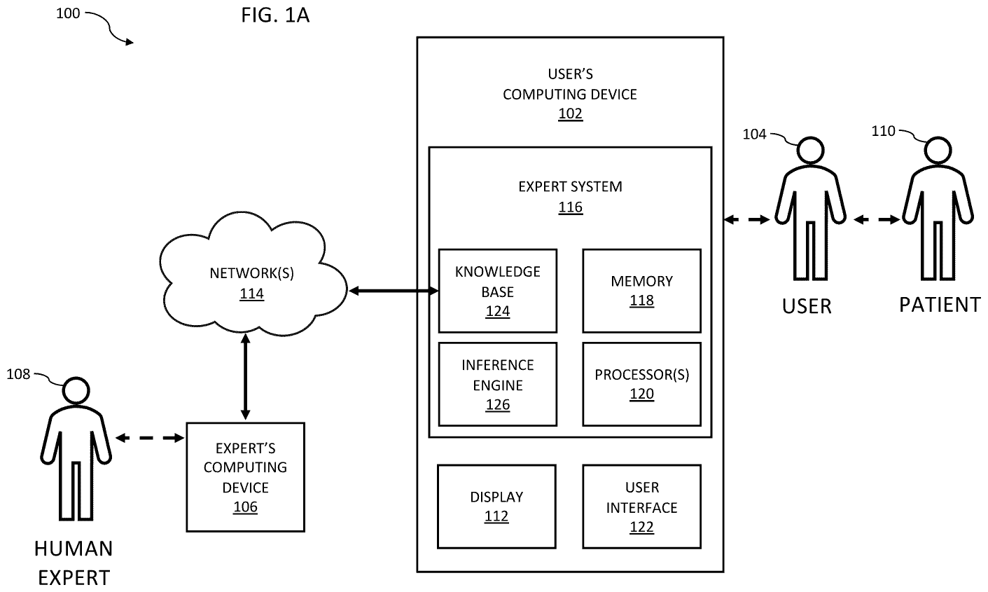

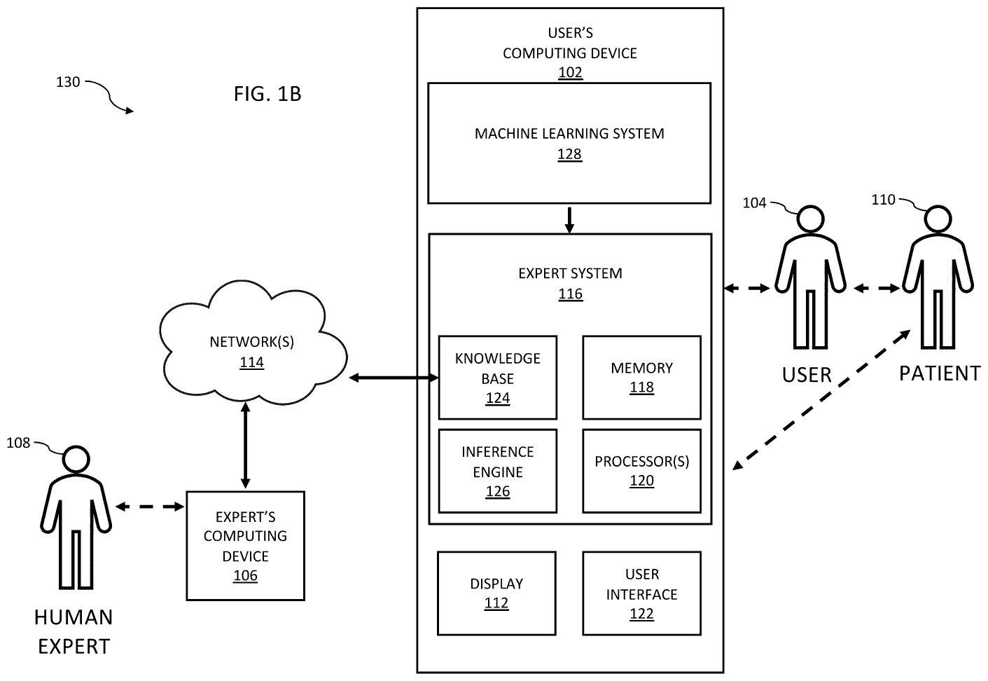

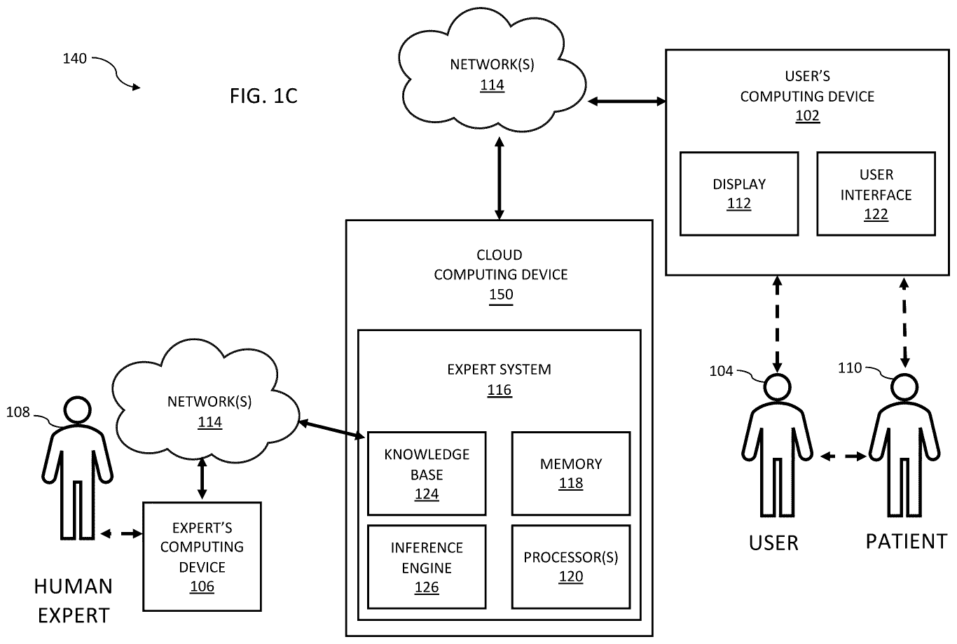

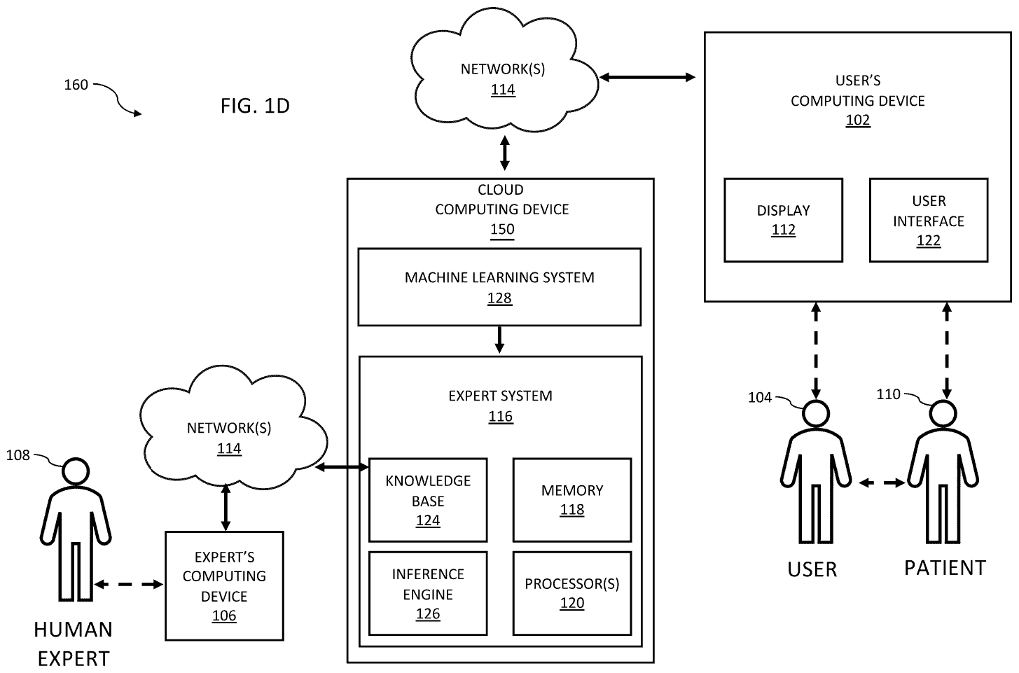

FIG. 1A illustrates a block diagram of a computing system 100 operable to construct an expert system 116 that runs on a user's computing device 102 to present expert system cosmetic dermatology treatment plans (such as laser settings or injectable medication plans) output to a user in accordance with embodiments of the present disclosure. Referring to FIG. 1A, the system 100 includes a computing device 102 of a user 104 and a computing device 106 of a human expert 108. In one example, the user 104 may be a dermatologist or non-dermatologist physician who has limited experience with laser treatments and would like to use the expert system 116 to receive recommended laser settings for treatment of a particular patient 110. In another example the user 104 may have limited experience with injectable medications for wrinkle relaxation and would like to use the expert system 116 to receive recommended injection sites and doses.

In one example, the human expert 108 may be a dermatologist who is an expert in laser treatments for skin conditions and/or an expert in injectable medication treatment for wrinkle relaxation. The human expert 108 may interact with the expert's computing device 106 to input information about the relationship between patient characteristics and laser settings, and/or to input information about the relationship between patient characteristics and injection locations and doses. This information may be transmitted via one or more network(s) 114 into an expert system knowledge base 124 of the expert system 116. This expert system knowledge base 124 is a structured representation of the relationship between patient characteristics and laser settings and/or injectable medication treatment plans. The expert system 116 includes and utilizes one or more memory units 118 and one or more processors 120.

The user 104 interacts with the patient 110 through an interview and/or through a physical examination to determine the patient's characteristics, which may include the patient's skin condition(s) or disease(s), Fitzpatrick skin type, wrinkle severity e.g., as measured on the Glogau Wrinkle Scale and/or Fitzpatrick Wrinkle Severity Scale, pigmentation e.g., as measured by the Kesty Pigmentation scale, redness e.g., as measured by the Kesty Redness scale, age, and/or face subunits needing treatment. The user 104 may interact with the computing device 102 to input patient characteristics via the user interface 122. For example, a display 112 of the computer device 102 may display to the user 104 a user interface 122 with input fields for specific patient characteristics, and the user 104 may fill in these input fields based on the information they gathered about the patient 110. The patient characteristics may be received and suitably stored at the computing device 102 or in the cloud. The patient characteristics may be sent into the expert system 116 and used by the inference engine 126 in conjunction with the knowledge base 124 in order to obtain predicted cosmetic dermatology treatment plans (such as recommended laser settings or recommended injection locations and doses). Recommended laser settings may include information about the laser brand, the energy, the pulse width, the spot size, the number of cooling passes, and/or miscellaneous configuration information. Recommended medication treatment plans may include, but is not limited to, information about the precise locations to inject medications along with the doses needed. A cosmetic dermatology treatment plan may be displayed to the user 104 via the display 112 of the user's computing device 102. The recommended cosmetic dermatology treatment plan may be determined by the expert system 116 on one or more computing devices.

FIG. 1B is a block diagram of a system 130 for constructing an expert system 116 and a machine learning system 128 that run on a user's computing device 102 to present cosmetic dermatology treatment plan output to a user in accordance with embodiments of the present disclosure. The description of FIG. 1B is the same as that of FIG. 1A with the one distinction that FIG. 1B additionally illustrates a machine learning system 128. The machine learning system 128 is trained to predict one or more skin features from one or more patient photographs. The user 104 or an assistant to the user 104 may take a photograph of the patient 110 and upload this photograph to the user's computing device 102. The machine learning system 128 then predicts skin features based on the provided patient photograph, and sends these predicted skin features to the expert system 116 as additional inputs. The expert system 116 may receive inputs from the machine learning system 128 and/or directly from the user 104. The purpose of including the machine learning system 128 is that this system 128 captures dermatologic expertise in the ability to predict skin features from a photograph, thus making it easier for the user 104 to utilize the expert system 116 because now the user 104 does not need to have the skill set necessary to predict the skin features from the patient's appearance. Instead, this dermatologic expertise is captured by the machine learning system 128.

FIG. 1C is a block diagram of a system 140 for constructing an expert system 116 that runs on a cloud computing device 150 to present cosmetic dermatology treatment plan output to a user in accordance with embodiments of the present disclosure. The description of FIG. 1C is the same as that of FIG. 1A with the one distinction that FIG. 1C illustrates a different computing configuration in which the expert system 116 runs on a cloud computing device (or server) 150 that interfaces via network(s) 114 with the user's computing device 102. The user's computing device 102 receives information on patient characteristics from the user 104 via the user interface 122, and then the user's computing device 102 transmits this information via network(s) 114 to the cloud computing device 150. The cloud computing device 150 runs the expert system 116 to predict laser settings based on the received patient characteristics. Then the output cosmetic dermatology treatment plan(s) are transmitted from the cloud computing device 150 through the network(s) 114 to the user's computing device 102 where they are displayed to the user 104 through the display 112.

FIG. 1D is a block diagram of a system 160 for constructing an expert system 116 and a machine learning system 128 that run on a cloud computing device 150 to present cosmetic dermatology treatment plan(s) to a user in accordance with embodiments of the present disclosure. FIG. 1D combines concepts from FIG. 1B and FIG. 1C. Like FIG. 1C, FIG. 1D includes a cloud computing device 150 that runs software to predict cosmetic dermatology treatment plan(s). However, FIG. 1D additionally includes the machine learning system 128 described in the description of FIG. 1B. Thus, in FIG. 1D, the cloud computing device 150 runs a machine learning system 128 to predict skin features from a patient photograph, and these skin features with or without additional patient characteristics provided by user 104 are the input to expert system 116 which outputs cosmetic dermatology treatment plan(s) that get displayed to user 104.

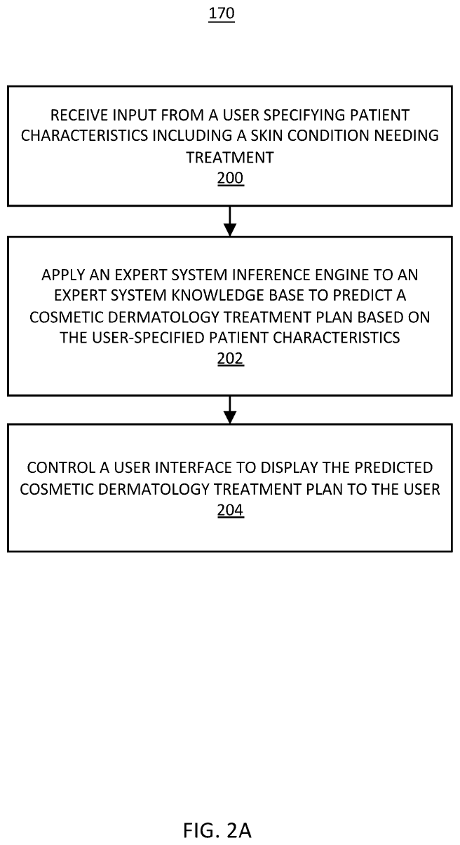

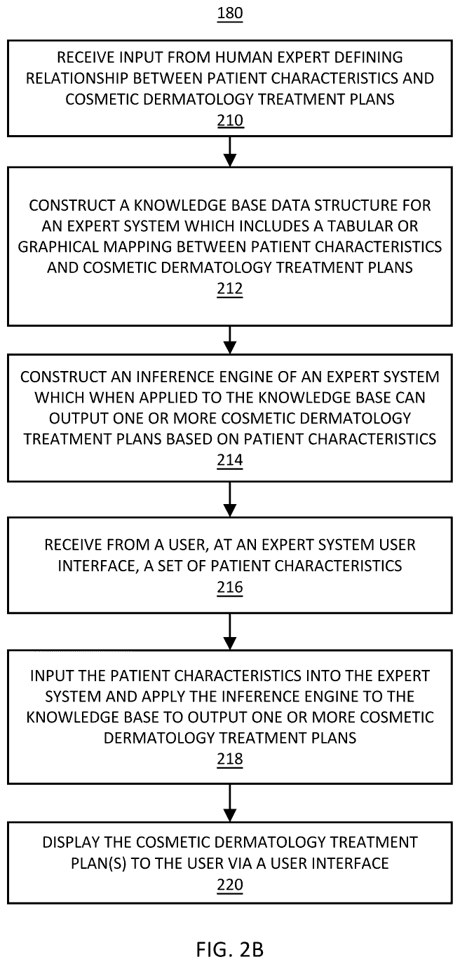

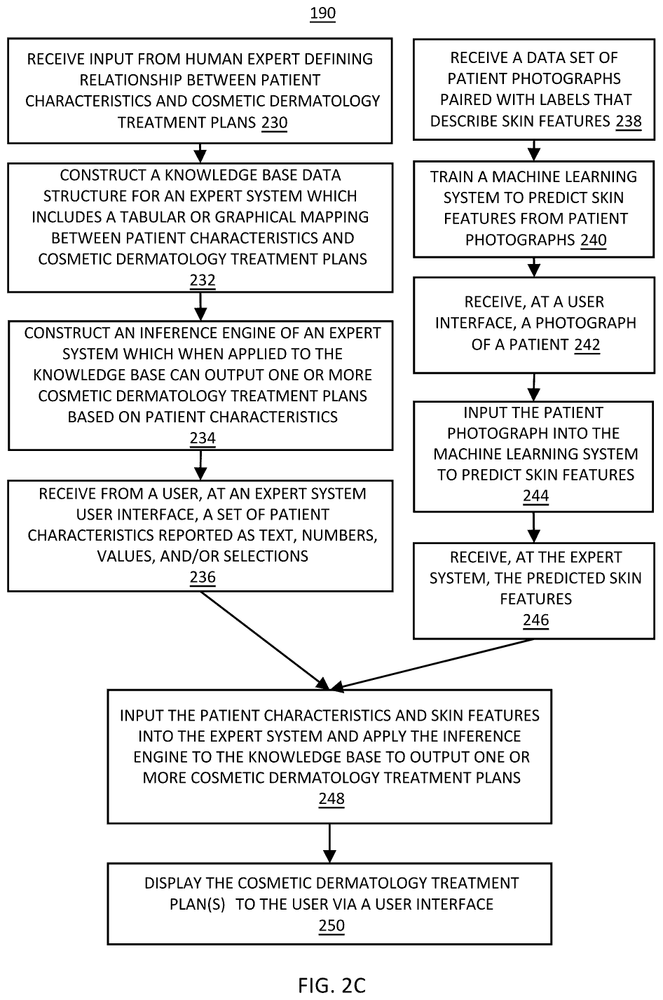

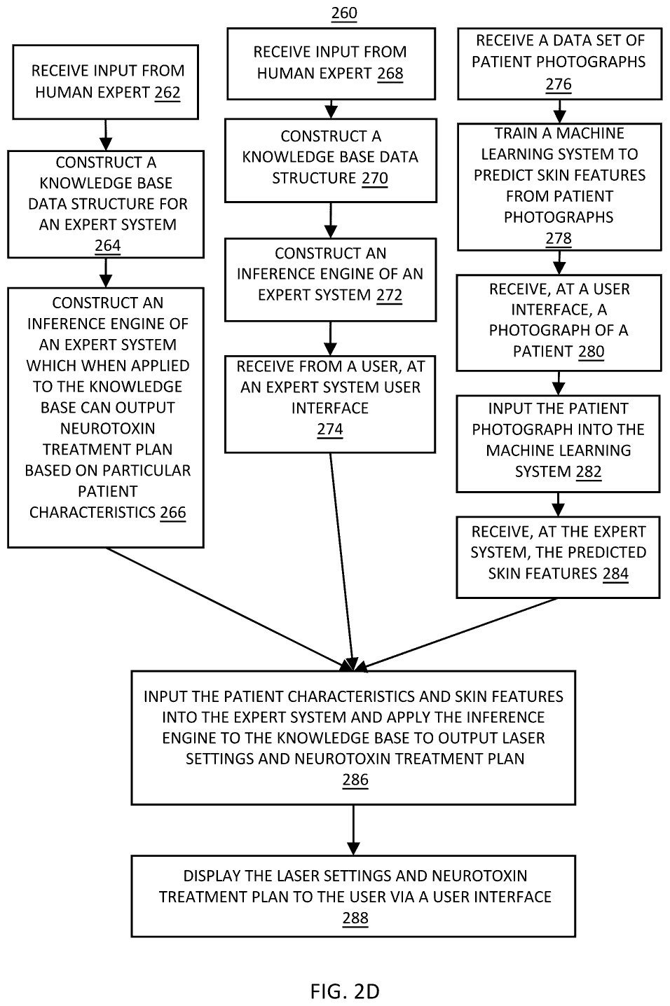

FIG. 2A is a flowchart of a method 170 for receiving user-specified patient characteristics for controlling display of a predicted cosmetic dermatology treatment plan in accordance with embodiments of the present disclosure. The method 170 may be implemented by one or more computing devices or systems, such as the example computing devices and system described herein. Referring to FIG. 2A, the method 170 includes receiving 200 input from a user specifying patient characteristics including a skin condition needing treatment.

The method 170 of FIG. 2A also includes applying 202 an expert system inference engine to an expert system knowledge base to predict a cosmetic dermatology treatment plan based on the user-specified patient characteristics. Further, the method 170 of FIG. 2A includes controlling 204 a user interface to display the predicted cosmetic dermatology treatment plan to the user.

FIG. 2B is a flowchart of a method 180 for constructing an expert system and for presenting the expert system cosmetic dermatology treatment plans to a user in accordance with embodiments of the present disclosure. Referring to FIG. 2B, the system may receive 210 input from a human expert such as a dermatologist that defines the relationship between patient characteristics and cosmetic dermatology treatment plans. The system may subsequently construct 212 a knowledge base data structure for an expert system. This knowledge base data structure may be a tabular data structure, tree data structure, graph data structure, set of if-then rules, or another format. The knowledge base indicates a mapping between patient characteristics and cosmetic dermatology treatment plans. The system may subsequently construct 214 an inference engine of an expert system which when applied to the knowledge base can output cosmetic dermatology treatment plan(s) based on particular patient characteristics. The inference engine may be different depending upon the structure of the knowledge base. If the knowledge base consists of if-then rules, then the inference engine will be configured to process if-then rules. If the knowledge base includes a tabular data structure, the inference engine may subsequently process tabular data. The system may subsequently receive 216 from a user at the expert system user interface a set of patient characteristics. Subsequently, the system may input 218 the patient characteristics into the expert system and apply the inference engine to the knowledge base to output cosmetic dermatology treatment plan(s) that are individualized based on the patient characteristics received. Subsequently, the system can display 220 the cosmetic dermatology treatment plan(s) to the user via a user interface.

FIG. 2C is a flowchart of a method 190 for constructing an expert system and a machine learning system that together enable presenting expert system cosmetic dermatology treatment plan(s) output to a user in accordance with embodiments of the present disclosure. The method 190 of FIG. 2C is similar to the method 180 of FIG. 2B except that the method 190 of FIG. 2C also includes a machine learning system that can predict skin features from a patient photograph. The predicted skin features are subsequently provided to the expert system as input.

Referring to FIG. 2C, the system may receive 230 input from a human expert such as a dermatologist that defines the relationship between patient characteristics and cosmetic dermatology treatment plans. The system may subsequently construct 232 a knowledge base data structure for an expert system. This knowledge base data structure may be a tabular data structure, tree data structure, graph data structure, set of if-then rules, or another format. The knowledge base indicates a mapping between patient characteristics and cosmetic dermatology treatment plans. The system may subsequently construct 234 an inference engine of an expert system which when applied to the knowledge base can output cosmetic dermatology treatment plan(s) based on particular patient characteristics. The inference engine may be different depending upon the structure of the knowledge base. If the knowledge base consists of if-then rules, then the inference engine will be configured to process if-then rules. If the knowledge base includes a tabular data structure, the inference engine may subsequently process tabular data. The system may subsequently receive 236 from a user at the expert system user interface a set of patient characteristics.

The system may receive 238 a data set of patient photographs paired with labels that describe skin features. The system may train 240 a machine learning system to predict the skin features from the patient photographs. The system may receive 242, at a user interface, a photograph of a patient. The system may input 244 the patient photograph into the machine learning system to predict skin features. The system may receive 246, at the expert system, the predicted skin features.

Subsequently, the system may input 248 the patient characteristics and skin features into the expert system and apply the inference engine to the knowledge base to output cosmetic dermatology treatment plan(s) that are individualized based on the patient characteristics and skin features received. Subsequently, the system can display 250 the cosmetic dermatology treatment plan(s) to the user via a user interface.

The method 260 of FIG. 2D is similar to the method 190 of FIG. 2C except that FIG. 2D explicitly illustrates how the system may be used to predict a neurotoxin treatment plan (injectable medication injection sites and doses) and/or laser settings.

Referring to FIG. 2D, for one expert system (e.g., for injectable medication injection sites and doses), the system may receive 262 input from a human expert. The system may subsequently construct 264 a knowledge base data structure for the expert system. The system may subsequently construct 266 an inference engine of the expert system which when applied to the knowledge base can output neurotoxin cosmetic dermatology treatment plan(s) based on particular patient characteristics.

For another expert system (e.g., for laser settings), the system may receive 268 input from a human expert. The system may subsequently construct 270 a knowledge base data structure for the expert system. The system may subsequently construct 272 an inference engine of the expert system which when applied to the knowledge base can output cosmetic dermatology treatment plan(s) (e.g., laser settings) based on particular patient characteristics. The system may subsequently receive 274 from a user at the expert system user interface a set of patient characteristics.

The system may receive 276 a data set of patient photographs paired with labels that describe skin features. The system may train 278 a machine learning system to predict the skin features from the patient photographs. The system may receive 280, at a user interface, a photograph of a patient. The system may input 282 the patient photograph into the machine learning system to predict skin features. The system may receive 284, at the expert system, the predicted skin features.

Subsequently, the system may input 286 the patient characteristics and skin features into the expert system and apply the inference engine to the knowledge base to output laser settings and neurotoxin treatment plan(s) that are individualized based on the patient characteristics and skin features received. Subsequently, the system can display 288 the laser settings and neurotoxin treatment plan(s) to the user via a user interface.

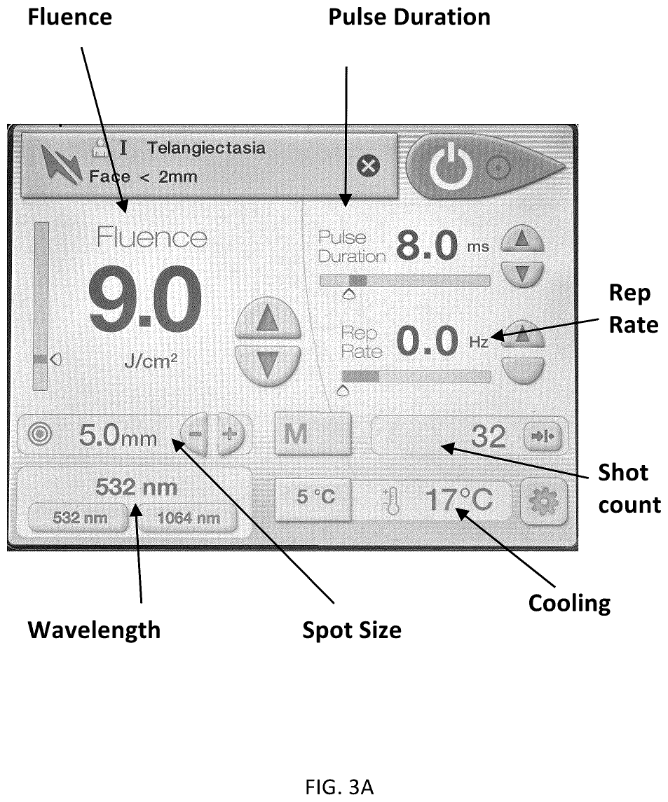

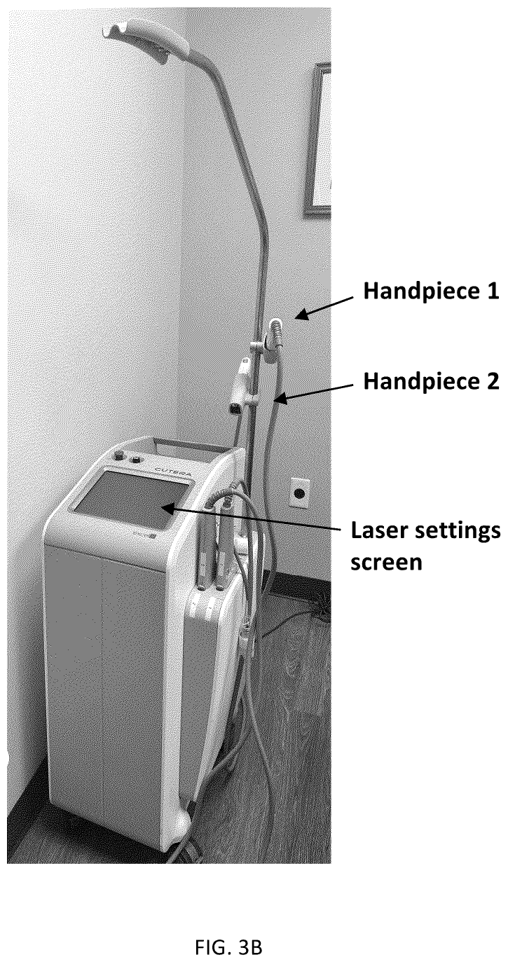

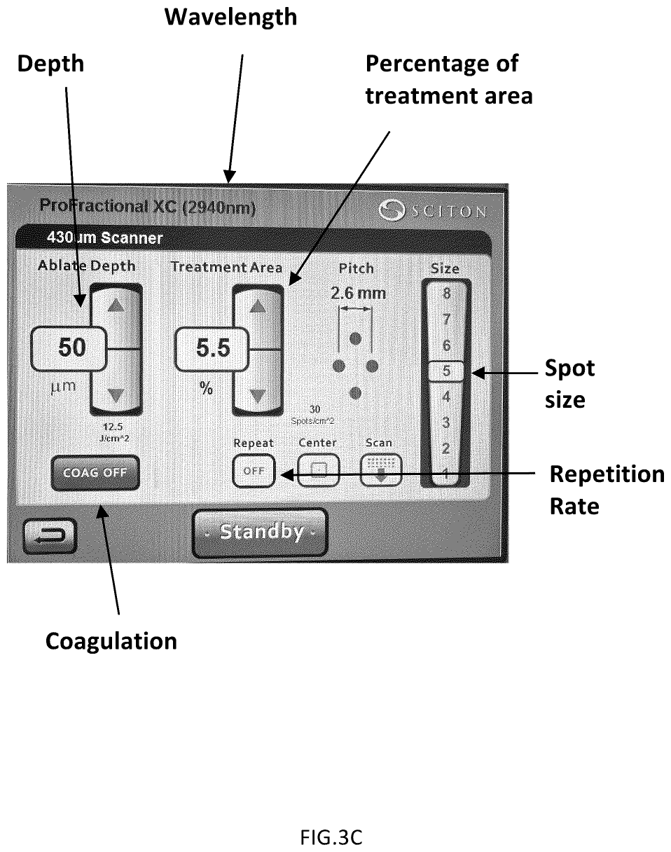

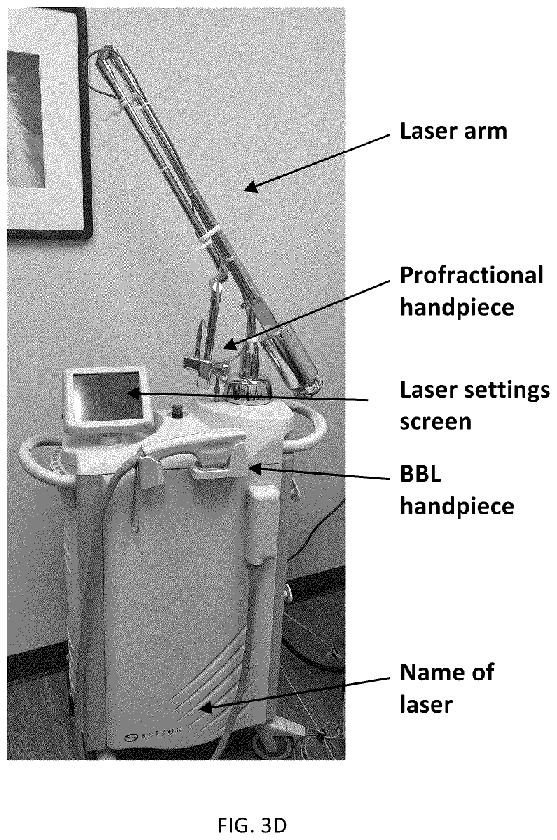

FIG. 3A is an example of a laser settings screen that a physician or non-physician provider can interact with to configure a particular laser, in this case the Cutera® Excel® V laser. This laser includes parameters including wavelength, fluence, pulse duration, repetition rate, shot count, spot size, and cooling. This is included as an example of the complexity of the configuration of an individual laser.

FIG. 3B is an example of the laser machine called a Cutera® Excel® V. This laser has both 532 and 1064 wavelengths and the configuration screen for the laser is shown in FIG. 3A.

FIG. 3C is the configuration screen to choose laser settings for the 2940 ProFractional Handpiece of the Sciton® Joule® Platform. Settings to choose and configure for this particular laser include the depth, wavelength, percentage of treatment area, spot size, repetition rate, and coagulation.

FIG. 3D is a second example of a laser machine, the Sciton® Joule®. This laser machine has 3 different wavelengths: 2940, 1064, and a Broadband Light handpiece. Each has unique configuration settings, one of which is shown in FIG. 3C.

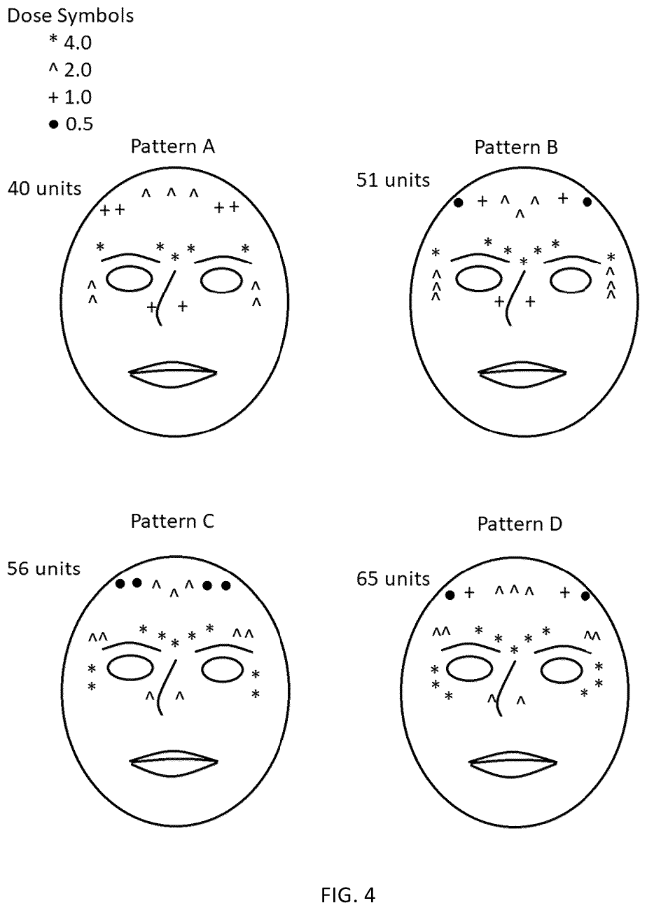

FIG. 4 is an example output of the presently disclosed subject matter, with specific doses of injectable neurotoxin as well as markers on the face of where each should be injected.

### Artificial Intelligence to Predict Cosmetic Dermatology Treatment Plan(s) Such as Therapeutic Laser Settings

The following sections describe different artificial intelligence approaches that may be leveraged to predict what cosmetic dermatology treatment plans to use in different clinical scenarios to achieve specific dermatologic outcomes. Clinical scenarios may vary according to patient characteristics, such as patient age and what condition is being treated.

The following sections primarily use prediction of therapeutic laser settings as an example. However, note that all of the AI systems described in the following sections could alternatively be applied to predict different kinds of cosmetic dermatology treatment plans as well; for example, treatment plans centered around use of injectable neurotoxic medications, in which treatment plans include injection locations and doses. Another example would be the acne severity score of the patient and treatment plans for their acne.

Prediction of cosmetic dermatology treatment plans may be achieved through use of an expert system (e.g., expert system 116). The expert system's user interface (e.g., user interface 122) may receive input directly from a human user, with or without additional input provided by a computer vision system that analyzes a patient photograph to predict skin characteristics. Prediction of cosmetic dermatology treatment plans may alternatively be achieved through use of a supervised machine learning model (e.g., machine learning system 128) trained end-to-end on tabular, text (including from a questionnaire), and/or image data.

### AI Expert System to Predict Laser Settings

An AI expert system (e.g., expert system 116) may be developed to predict what therapeutic laser settings to use to achieve a desired outcome. An expert system is a form of artificial intelligence that leverages human knowledge in order to solve a complex problem. Expert systems may have multiple components including a knowledge base (e.g., knowledge base 124), an inference engine (e.g., an inference engine 126), and a user interface (e.g., user interface 122). The knowledge base stores a representation of human expert knowledge, while the inference engine is applied to the knowledge base to provide answers and suggestions in a manner similar to a human expert. The user interface allows users to interact with the system without computer programming.

### AI Expert System: Knowledge Base

The knowledge base (e.g., knowledge base 124) may be represented in the form of rules, or the knowledge base may be represented in the form of frames, which are data structures that relate an object/item/concept to a collection of facts and/or values. Examples of frames may include a tree data structure and a 2D tabular data structure.

An expert system (e.g., expert system 116) to predict laser settings may leverage a series of if-then rules as its knowledge base. For example, the rules may be structured as follows: if ((patient needs treatment for condition X) AND (patient is of age Y) AND (the part of the face requiring treatment is Z) AND (patient's skin type is Q)) then ((laser type is A) AND (laser settings are B, C, D)).

An expert system (e.g., expert system 116) to predict laser settings may also leverage a tree data structure as its knowledge base, where the tree data structure is a type of frame. There may be branch points in the tree based on type of condition, patient's age, part of the face requiring treatment, patient's skin type, and other patient characteristics summarized in Table 1. The leaves of the tree may contain laser settings corresponding to a particular combination of the aforementioned factors.

Finally, an expert system (e.g., expert system 116) to predict laser settings may leverage a 2D tabular data structure as its knowledge base, where the tabular data structure is a type of frame. This table may include, for example, the columns described in Table 1 below.

TABLE 1
Summary of columns of a 2D tabular data structure leveraged for laser setting
prediction. “Column Name” is a name summarizing the column, “Column
Description” is a detailed description of the column's meaning, and “Column
Type” indicates whether the column is describing patient characteristics or laser settings:

| Column Name | Column Description | Column Type |
|---|---|---|
| Skin | This column can specify the type of skin condition that | Patient |
| Concern | needs to be treated using laser therapy and/or the goal | Characteristics |
| | of the treatment. Skin conditions or goals of treatment | |
| | may include: fine lines/wrinkles/crepey/overall | |
| | rejuvenation, loose/sagging skin, acne/rosacea, | |
| | scarring, vascular (red telangiectasia), vascular (blue | |
| | veins), vascular (cherry angioma), pre-cancers, eye | |
| | rejuvenation/tightening, perioral wrinkles, seborrheic | |
| | keratoses (raised), seborrheic keratoses (flat), freckles, | |
| | lentigos, eye rejuvenation/tightening, perioral wrinkles | |
| Fitzpatrick | Indicates the patient's Fitzpatrick skin type. | Patient |
| skin type | Fitzpatrick Skin Type (1-6) | Characteristics |
| | Type 1 (most white/burns most easily) | |
| | Type 2 | |
| | Type 3 | |
| | Type 4 | |
| | Type 5 | |
| | Type 6 (most dark) | |
| Glogau | Glogau Wrinkle Scale (1-4) | Patient |
| Wrinkle | Type 1: No Wrinkles | Characteristics |
| Scale | Type 2: Wrinkles In Motion | |
| | Type 3: Wrinkles At Rest | |
| | Type 4: Only Wrinkles | |
| Kesty | Kesty Pigmentation (0-3) | Patient |
| Pigmentation | 0 = none | Characteristics |
| | 1 = mild | |
| | 2 = moderate | |
| | 3 = severe | |
| Kesty | Kesty Redness (0-4) | Patient |
| Redness | 0 = clear | Characteristics |
| | 1 = almost clear | |
| | 2 = mild | |
| | 3 = moderate | |
| | 4 = severe | |
| Fitzpatrick | Fitzpatrick Wrinkle Severity Scale (1-9) | Patient |
| Wrinkle | 1 = least wrinkles | Characteristics |
| Severity | 9 = most wrinkles | |
| Scale | | |
| Age | The patient's age | Patient |
| | | Characteristics |
| Face | The part of the face that requires treatment. The location | Patient |
| Subunit | may be specified in a general way (e.g. “forehead”) or | Characteristics |
| and/or | in an extremely specific way (e.g. by an annotation | |
| Location | drawn directly on a photograph of the patient's face). | |
| of Skin | | |
| Concern | | |
| Name of | The brand of laser and/or specification of the exact | Laser Settings |
| laser | model (including wavelength of the laser to be used) | |
| Fluence | The energy per unit area (joules per centimeter | Laser Settings |
| | squared) of the absorbing surface (the skin) | |
| Pulse | The pulse width is also sometimes referred to as the | Laser Settings |
| width | pulse duration and indicates the length of time of the | |
| | pulse. | |
| Spot size | The radial distance of the laser's beam, which affects | Laser Settings |
| | the penetration depth of the laser. | |
| Cooling | Specification of the skin cooling needed during | Laser Settings |
| | treatment | |
| Percentage | The percentage (%) of the skin surface that is affected | Laser Settings |
| of | by the Laser beam (ranges from 0-100%) | |
| Treatment | | |
| Area | | |
| Depth | Depth of the Laser beam penetration into the skin | Laser Settings |
| Coagulation/ | The level of coagulation or added heat that is in | Laser Settings |
| Added | addition to the wavelength of the laser | |
| Heat | | |
| Laser notes | Any miscellaneous notes about laser configuration that | Laser Settings |
| | may be helpful to a clinician | |

In a variation of the above table, the “Laser Settings” columns may be represented multiple times, once for each different kind of laser that may be considered. Thus, for a single combination of patient related values, there may be multiple appropriate lasers each with their own settings.

### AI Expert System Inference Engine

The inference engine (e.g., an inference engine 126) of the expert system (e.g., expert system 116) is applied to the knowledge base (e.g., knowledge base 124) in order to output recommended laser settings. The inference engine may be implemented in different ways. The inference engine may take as input a specification of one individual patient's characteristics. For if-then rules as the knowledge base, the inference engine may traverse the rules to identify rules related to that patient's characteristics, in order to determine the laser settings. For a tree data structure as the knowledge base, the inference engine may traverse the tree structure, at every branch point comparing the individual patient's characteristics with the attributes in the tree structure to determine which path to take through the tree. For a tabular data structure as the knowledge base, the inference engine may compare the individual patient's characteristics with the values in the corresponding patient characteristics columns in order to identify a row that most closely matches the individual patient, which then in turn contains appropriate laser settings.

The inference engine may be implemented in a modern programming language such as JavaScript, TypeScript, or Python.

### AI Expert System: User Interface

The expert system (e.g., expert system 116) may have an associated user interface (e.g., user interface 122). The user interface may include fields corresponding to patient characteristics where a user may enter details corresponding to an individual needing treatment. The user interface may also include a place to display output, for example displaying recommended laser settings. Patient characteristics input fields may be implemented as text boxes that accept numeric input, text boxes that accept text input, button selections including radio buttons, and/or dropdown menus that may or may not be searchable. The expert system may also incorporate a questionnaire based on text to input patient characteristics from a series of questions that are answered by the patient or a provider.

The expert system may also have a separate associated user interface for entering expert knowledge. The user interface for entering expert knowledge may include text boxes, a tree data structure, and/or a tabular data structure.

### AI Expert System: Tabular Data Structure Knowledge Base Creation

An initial version of the tabular data knowledge base (e.g., knowledge base 124) may be created by a board-certified dermatologist using software such as Google Sheets or Excel. The final version of the tabular data knowledge base may be created by a programmer or engineer who restructures, cleans, or reorganizes the dermatologist-created file into a form that can be easily consumed by a software program.

Machine Learning Model to Predict Fitzpatrick Skin Type, Glogau Wrinkle Scale, Kesty Pigmentation, Kesty Redness, and/or Fitzpatrick Wrinkle Severity Scale

The expert system (e.g., expert system 116) previously described may receive input from a human user via a user interface (e.g., user interface 122). The expert system may additionally receive some input from a machine learning model (e.g., machine learning system 128) that predicts skin characteristics such as Fitzpatrick Skin Type, Kesty Pigmentation, Kesty Redness, and/or Fitzpatrick Wrinkle Severity Scale. The purpose of using a machine learning model to predict these skin features is to enable use of the expert system in circumstances where the human user does not have the dermatologic training necessary to accurately input the patient's skin features. The machine learning model can capture dermatologic expertise and automatically predict the patient's skin features, enabling use of the expert system by for example doctors who have minimal dermatology background.

Furthermore, this machine learning model may also be useful in future clinical research applications, because the skin features listed above are standardized scales often used in research. Thus there could be multiple potential research applications of a machine learning model that can predict these standardized skin scales from patient photographs. For example, some research projects could be conducted more quickly and cheaply if skin features were determined for study participants automatically using a machine learning model rather than manually by a human expert. Also, the results of lasers to treat dermatologic conditions could also become more standardized if the scales that proved laser results were more standard across studies. For example, if the machine learning algorithm classifies each before and after photos for 2 different lasers studies, the results from each study may be compared. On the other hand, if two different humans classified the faces of before and after photos for each study, then they may choose different values for each scale and therefore the results may not be compared as efficiently. The proposed inventions may be useful in supporting future research on lasers and energy-based therapeutic devices.

Constructing the machine learning model to predict skin characteristics may involve different techniques for dataset creation, model architecture, and model training.

### Machine Learning to Predict Skin Characteristics: Dataset Creation

Dataset creation may involve publicly available images and/or images collected in a clinical setting.

Publicly available images may be obtained from sources like Wikipedia/Wikimedia. Images may be filtered to select only those licensed under “Creative Commons” licenses or those listed as being in the public domain. Images may depict humans of all ages, genders, races, ethnicities, and demographic backgrounds in order to create a diverse dataset. Images may be filtered to include only those images which depict the face or a certain minimum percentage of the face, for example at least 80% of the face. The images must show the subject's skin, as this will be needed for skin type classification.

In one example, a dataset was created using publicly available images from Wikimedia licensed as Creative Commons or public domain. Images were selected such that the resulting dataset comprised approximately 50% male individuals and 50% female individuals spanning a range of ages, from young adults (e.g., ˜18 years old) to the elderly (e.g., ˜90 years old). The dataset was also crafted to include approximately ⅓ White individuals, ⅓ Asian individuals including Southeast Asia/India, and ⅓ Black individuals including African Americans. Only images depicting at least 70% of the subject's face were included. Images were taken in a variety of lighting conditions, including both outdoor and indoor settings. Subjects were wearing a variety of clothing articles including Western and non-Western clothing styles.

The images may be labeled by one or more board-certified dermatologists according to the following scales: Fitzpatrick Skin Type, Kesty Pigmentation, Kesty Redness, Fitzpatrick Wrinkle Severity Scale, and/or any other scales or rating systems commonly applied to skin by dermatologists.

The labels may be acquired through the interface of an online labeling service such as LabelBox. In an example, one board-certified dermatologist interacted with LabelBox software to apply the aforementioned labels to every image in the dataset.

If labels from multiple dermatologists are acquired, a consensus label can be determined using any of the following approaches, for each labeling scale separately:
The label most commonly selected can be chosen (majority vote);
An average can be taken of all the labels with or without rounding to the nearest integer;
The labelers can discuss that individual photo to reach a consensus.

### Machine Learning to Predict Skin Characteristics: Dataset Preprocessing

Before images are leveraged to train or validate a machine learning system (e.g., machine learning system 128), the dataset may be preprocessed using a variety of techniques, including but not limited to:
Converting the image file format into a format compatible with major machine learning frameworks—for example, converting a PNG or JPEG file into a NumPy array compatible with the PyTorch and TensorFlow machine learning frameworks;
Leveraging a pretrained object detection system to identify the human face and crop the image to include only the human face;
Normalizing and centering pixel values based on summary statistics such as the mean and standard deviation of the pixel values in the training dataset, for each color channel separately.

Data augmentation has been shown to increase the performance of computer vision models across numerous application areas. Data augmentation procedures may be employed, including but not limited to:
Random flips across the vertical and/or horizontal directions;
Random resizing/scaling changes;
Random rotation;
Random crops.

Data augmentation may leverage an existing library or package for this purpose, such as,
scikit-image (https://scikit-image.org/)
Augmentor (https://augmentor.readthedocs.io/en/master/)
Albumentations (https://albumentations.ai/)
Torchvision (https://pytorch.org/vision/stable/index.html).

Color transformations should not be used for data augmentation because some of the scales such as the Fitzpatrick skin type are dependent upon color.

### Machine Learning to Predict Skin Characteristics: Model Training and Evaluation

Different machine learning models (e.g., machine learning system 128) may be trained to predict skin characteristics from images. Models may include neural network or deep learning based architectures such as convolutional neural networks (CNNs) or Vision Transformers (ViT) or models that combine aspects of convolution and attention. Convolutional neural networks are a type of neural network that learns features via filter optimization. Vision Transformers are a type of Transformer architecture targeted towards computer vision tasks. Vision transformers break images down into patches which are transformed into vectors and then processed using a mechanism called attention.

The machine learning model (e.g., machine learning system 128) to predict skin characteristics may be pre-trained on another dataset of natural images and/or other medical images. For example, the model may be pre-trained on ImageNet images. After pre-training the model may have weights in early layers fixed and weights in later layers fine-tuned on patient images. Alternatively, all of the weights in the model may be fine-tuned on patient images.

A single model (e.g., machine learning system 128) may be trained to predict multiple skin characteristics simultaneously. For example, one model may be trained to simultaneously predict Fitzpatrick Skin Type, Glogau Wrinkle Scale, Kesty Pigmentation, Kesty Redness, and/or Fitzpatrick Wrinkle Severity Scale. Prior work in machine learning on CT scans has shown that training one model to predict multiple outputs from the same image can yield higher performance than training multiple separate models, one for each output. Thus, it is anticipated that training one model to predict numerous skin characteristics will yield higher performance.

Neural network models are “black box” models by default, meaning that they do not provide any explanations of how they made their predictions. Prior work in machine learning has shown that models can exploit spurious correlations to increase performance—for example predicting boats through the presence of water, or predicting pneumonia in a chest x-ray by the presence of metal tokens that are correlated with pneumonia prevalence by happenstance. In sensitive domains such as healthcare, it is especially important to gain insight into how models make predictions, to ensure that only medically reasonable models are deployed.

Thus, explainability techniques for neural network models may be incorporated. For example, the HiResCAM technique may be included. In this technique, a convolutional neural network model must end in only one fully connected layer, and then HiResCAM is applied to create a colormap showing which part of the input image was used to make a particular prediction.

### Using Machine Learning Instead of an Expert System to Predict Laser Settings

The previous sections described how an expert system (e.g., expert system 116) may be used to predict laser settings based on input from a human expert and/or input from a machine learning model (e.g., machine learning system 128). In an alternative approach, a fully machine learning based framework (e.g., machine learning system 128) may be used to predict laser settings. Supervised machine learning methods may be employed for this purpose, for example neural networks (such as multilayer perceptrons or deep neural networks), support vector machines, regression, random forests, Naive Bayes classifiers, and/or an ensemble of any of the aforementioned methods. The data to train and evaluate the laser prediction machine learning models may be obtained through clinical research studies designed for data collection purposes or through electronic medical records and may include any of the following elements:
Photographs of patient's face;
Structured or unstructured (e.g., free text) descriptions of the patient's reason for needing laser treatment;
Structured or unstructured descriptions of the laser settings applied to the patient;
Structured or unstructured descriptions of the patient's cosmetic outcome and whether the desired outcome was achieved.

Appropriate consent from patients for use of their data will be obtained as necessary.

Identifiable data may be analyzed within a protected computing environment that complies with HIPAA requirements for patient privacy. Data may also be de-identified/anonymized and then analyzed within any computing environment.

Data de-identification may include any of the following steps:
Anonymization of photographs by irreversibly blacking out (setting to black color) any pixels corresponding to particularly identifiable parts of the human face, such as the eyes;
Anonymization of structured data by removing any identifiers that were part of the patient's official electronic health record and replacing these identifiers with a different randomly-generated identifier that contains no protected health information;
Stripping dates from the dataset;
Anonymization of free text data through creation of a whitelist of allowed vocabulary that excludes any protected health information, and then processing all free text data to replace with “XXX” or a similar token any words not found in the whitelist. The whitelist may be created by first obtaining a list of all unique words found in the free text data and then removing any words that indicate protected health information (e.g., names). The whitelist may also be created by using publicly available whitelists. This whitelist can also help with date stripping.

Data cleaning may include any of the following steps:
Standard pre-processing of photographs to scale, center, and/or normalize the pixel values;
Standardization of any coding systems used—for example updating ICD-9 codes to ICD-10 codes;
Standardization of medication information e.g. by mapping to active ingredients represented as generic names;
Text cleaning including replacement of all whitespace with a single space; removal of most punctuation except for periods, hyphens, or parentheses; and lowercasing.

Machine learning models (e.g., machine learning system 128) may be trained on cleaned data that may or may not have been anonymized. The input to the machine learning models may include patient characteristics described in Table 1 as “Patient Characteristics,” while the output of the machine learning models may include the laser settings described in Table 1 as “Laser Settings.” There are multiple possible configurations for model training, including:
Train one model on image(s) of a patient's face to predict skin characteristics, and use its output as part of the input to a separately trained model that uses patient characteristics including patient age and conditions to predict laser settings.
Train a single end-to-end model that takes as input both image(s) of a patient's face as well as other patient characteristics such as age and conditions, to predict laser settings.

### Artificial Intelligence to Predict an Injectable Medication Treatment Plan for Wrinkle Relaxation

Artificial intelligence may also be used to predict a treatment plan for using injectable medications for wrinkle relaxation. The treatment plan for injectable medications may include prediction of what doses to use, prediction of the injection points, or a combination thereof. The AI system may include an expert system (e.g., expert system 116) and/or a machine learning system (e.g., machine learning system 128).

### Artificial Intelligence System to Predict Doses Only for Wrinkle Relaxation

In one example, an AI system may be developed to predict the doses of injectable medication needed for wrinkle relaxation.

TABLE 2
Injection muscle targets for wrinkle relaxation. This table
includes an example of how injection sites may be described
in words as part of AI systems that predict dose only.

Lateral frontalis 1 cm above lateral eyebrow
Upper frontalis
Corrugator supercilli
Procerus
Nasalis muscle
Orbicularis oculii
Orbicularis oris
Mentalis
Depressor Anguli oris

TABLE 3
Examples of injectable medications. This table lists the names
of injectable medications that may be included in an AI system
for predicting cosmetic dermatology treatment plans.

Botox ®
Dysport ®
Jeaveau ®
Xeomin ®
Daxxify ®
Letybo ®

In an expert system implementation of dose-only prediction, an expert system knowledge base (e.g., knowledge base 124) may be constructed to capture the mappings between:
patient characteristics such as those listed in Table 1, including the face subunit/anatomical location requiring treatment. The anatomical location may optionally be described via selection of any of the injection sites shown in Table 2.
dosing amounts for any or all locations shown in Table 2 and for any or all medications shown in Table 3.

A separate dosing amount mapping may be created for each separate injectable medication.

An expert system inference engine (e.g., inference engine 126) may be applied to the knowledge base (e.g., knowledge base 124) such that the expert system (e.g., expert system 116) can receive as input a group of patient characteristics, and then produce as output one or more medications (Table 3) for one or more locations (Table 2) associated with one or more dosing amounts. The output is a treatment plan for wrinkle relaxation. The output may be formatted as a list of dosing amounts for each location (Table 2) that is relevant to the patient based on the patient's characteristics.

In a machine learning implementation of dose-only prediction, a machine learning model (e.g., machine learning system 128) may be trained to predict dosing from patient characteristics. A dataset is created that includes examples of many patients, where each patient has multiple characteristics recorded (e.g., patient characteristics shown in Table 1). The dataset is then labeled with a dose amount for each of the locations shown in Table 2 and each of the injectable medications shown in Table 3, where one injectable medication may have entirely different dose amounts than another injectable medication. A machine learning regressor, for example implemented using logistic regression or a neural network, is trained on the dataset to receive patient characteristics as input and predict as output the dose amounts for different locations and/or injectable medications.

### Artificial Intelligence System to Predict Injection Points Only for Wrinkle Relaxation

A machine learning system (e.g., machine learning system 128) may be implemented to predict injection points for wrinkle relaxation. A dataset may be created including photographs of patient faces. The dataset may be labeled with point annotations, line annotations, or bounding box annotations that indicate a location on the face that should receive an injection. A machine learning model (e.g., machine learning system 128) may be trained on the dataset to receive as input a photograph of a patient's face and predict as output the points, lines, or bounding boxes that visually indicate where injectable medication should be injected in order to achieve wrinkle relaxation.

### Artificial Intelligence System to Predict Injection Points and Doses for Wrinkle Relaxation

An artificial intelligence system may be implemented to predict injection points and doses simultaneously.

A machine learning system (e.g., machine learning system 128) like that described above for injection point prediction may be applied to predict injection points from photographs such that the injection points may be visually displayed over the patient photograph. Then, an expert system (e.g., expert system 116) or machine learning system (e.g., machine learning system 128) like those described for dose prediction only may be applied in order to output dosing information for each of the predicted injection points, where the system relies upon a naming scheme for injection points such as that shown in Table 2.

Alternatively, an end-to-end machine learning system (e.g., machine learning system 128) may be created that predicts injection points and doses simultaneously. A dataset may be created in which patient face photographs are annotated with point annotations, line annotations, or bounding box annotations, and in addition each of the points, lines, or boxes is further associated with (a) the names of one or more injectable medications and, (b) for each injectable medication, the dosing information. An end-to-end machine learning system (e.g., machine learning system 128) is then trained to receive a patient face photograph as input and predict as output the injection points and associated medication/dosing information.

Artificial Intelligence System to Match Patients with Predefined Injection Point/Dose Patterns

An artificial intelligence system may be created to match patients with predefined injection point and dose patterns.

A set of predefined injection point and dose patterns may be defined. For example, FIG. 4 shows examples of injection point and dose patterns, in which injection points are drawn on a generic representation of the human face, and different doses are visually indicated using different symbols such as an asterisk, triangle, plus sign, or point. Injection point and dose patterns may be defined with a hand drawing, a digital drawing, a 3D rendering, or any other mechanism. Different doses may be indicated using symbols as shown in FIG. 4 or the doses may also be written on the diagram next to the associated injection point. Each injection point/dose pattern may be assigned a unique identifier, such as “0001” or “ABCDE” or “ABC123” in order to be able to refer to that unique injection point/dose pattern easily within a computer program.

An AI expert system knowledge base (e.g., knowledge base 124) may be defined that includes a mapping between a wrinkle severity score and a particular injection point/dose pattern that would be suitable for achieving wrinkle relaxation for that patient based on their wrinkle severity score. The injection point/dose pattern may be referred to by its unique identifier as shown in Table 4.

TABLE 4
An example of an expert system knowledge base mapping between
wrinkle severity and injection point/dose patterns. The
wrinkle severity shown here is indicated by the Fitzpatrick
Wrinkle Severity Scale but in practice any measuring scale
for wrinkle severity may be used. The injection point/dose
patterns are referred to only by different identifiers meant
to indicate different patterns. A particular wrinkle severity
score may correspond to one or multiple patterns. Also,
multiple different wrinkle severity scores may correspond
to the same pattern. This table is only one example of how
such a mapping could be constructed.

| Fitzpatrick Wrinkle | Injection Point/Dose Pattern Identifier |
|---|---|
| Severity Scale | (Conceptual Example) |
| 1 | AAA0001 |
| 2 | AAA0002 |
| 4 | AAA0003 |
| 5 | AAA0004 |
| 6 | BBB001 |
| 7 | BBB0002 |
| 8 | CCC0001 |
| 9 | CCC0002 |

The expert system (e.g., expert system 116) may receive as input a patient's wrinkle severity score. The expert system inference engine (e.g., inference engine 126) may be applied to the knowledge base (e.g., knowledge base 124) in order to select an injection point/dose pattern identifier that is an appropriate cosmetic dermatology treatment plan based on the wrinkle severity score. This injection point/dose pattern may be displayed to a user via a user interface. It may be displayed in a graphical manner such as via a face diagram with annotations like those shown in FIG. 4.

As a further example, a machine learning model (e.g., machine learning system 128) may be used to predict wrinkle severity from a patient face photograph, as described earlier in this application. The predicted wrinkle severity score may then be provided to the expert system (e.g., expert system 116), so that the expert system can predict an injection point/dose pattern that will be a suitable treatment.

As a further example, an application may be developed in which a patient can align their own face with a digital mask. The patient may align their face with the digital mask based on anatomical features such as eyes, eyebrows, nose, mouth, and chin. A digital image may be stored that includes the photograph of the patient's face along with the superimposed digital mask in the position that the patient aligned it, and further including injection points that are part of the definition of the digital mask. This digital image may be shown to a user of an AI system as an illustration of where injection points should be located relative to an individual patient's face.

Artificial Intelligence System that Automatically Configures Cosmetic Dermatology Equipment, Predicts Acne Severity and Recommends Acne Care Products and Treatments, Implements a Feedback Loop, and Provides a Virtual Patient Portal Dashboard

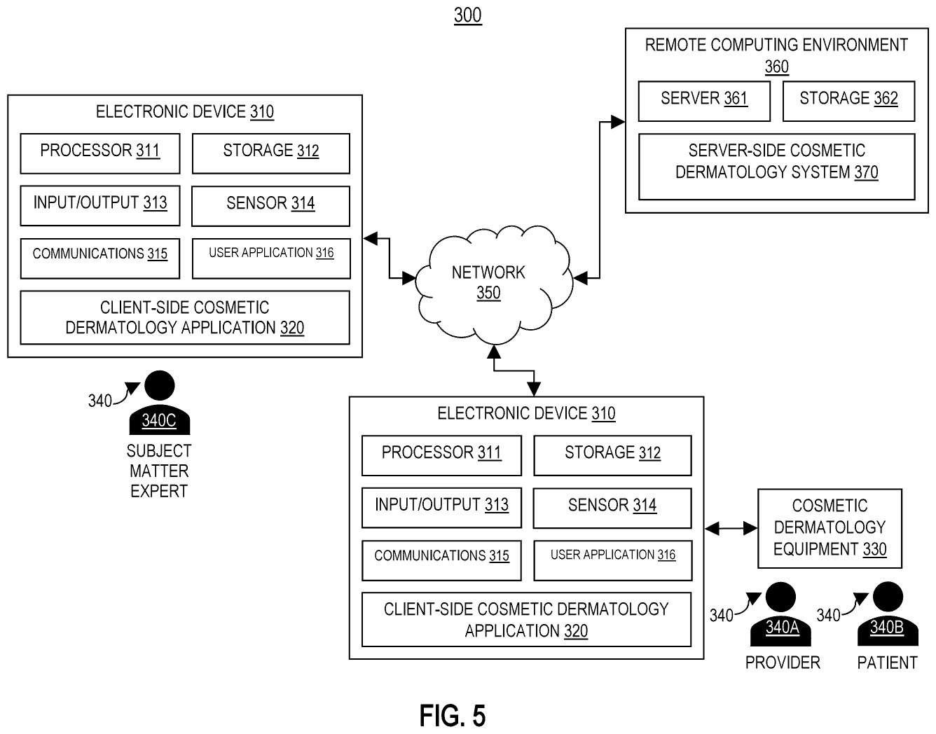

FIG. 5 is an example computing architecture 300 for an AI cloud platform for cosmetic dermatology, in one or more embodiments. The computing architecture 300 comprises at least one electronic device 310. Each electronic device 310 includes resources, such as one or more processor units 311 and one or more storage units 312. Applications may execute/operate on an electronic device 310 utilizing resources of the electronic device 310.

Examples of an electronic device 310 include, but are not limited to, a mobile electronic device (e.g., an optimal frame rate tablet, a smart phone, a laptop, etc.), a wearable device (e.g., a smart watch, a smart band, a head-mounted display, smart glasses, etc.), a desktop computer, a gaming console, an Internet of things (IoT) device, etc.

In one embodiment, applications on an electronic device 310 include one or more user applications 316 loaded onto or downloaded to the electronic device 310, such as a camera application, a social media application, a web browser, etc.

In one embodiment, applications on an electronic device 310 further include a client-side cosmetic dermatology application 320. In one embodiment, a client-side cosmetic dermatology application 320 on an electronic device 310 is configured to interface and exchange data with a user application 316 on the same electronic device 310, such as invoking a camera application on the electronic device 310 to capture a photo.

In one embodiment, a client-side cosmetic dermatology application 320 on an electronic device 310 is configured to interface and exchange data with a cosmetic dermatology equipment 330. In one embodiment, a cosmetic dermatology equipment 330 is a standalone device that is coupled to an electronic device 310. For example, in one embodiment, a cosmetic dermatology equipment 330 and an electronic device 310 are connected via a wired connection (e.g., physical LAN cable, etc.), a wireless connection (e.g., Bluetooth®, Wi-Fi, cellular data, etc.), or a combination of both a wireless connection and a wired connection. In another embodiment, a cosmetic dermatology equipment 330 is integrated into, or implemented as part of, an electronic device 310.

Examples of a cosmetic dermatology equipment 330 include, but are not limited to, a therapeutic laser (i.e., laser therapy device), a patient photography device, or clinical/medical imaging device, a spa treatment device, an exfoliation device, etc.

In one embodiment, an electronic device 310 comprises one or more input/output (I/O) units 313 integrated in or coupled to the electronic device 310. In one embodiment, the one or more I/O units 313 include, but are not limited to, a local physical user interface (PUI) and/or a graphical user interface (GUI), such as a remote control, a keyboard, a keypad, a touch interface, a programmable logic controller (PLC) user interface, a touch screen, a knob, a button, a display screen, etc. In one embodiment, a user 340 can utilize at least one I/O unit 313 (e.g., a PUI or GUI) to configure one or more configuration settings, provide user input (e.g., user preferences, user feedback), etc.

Examples of a user 340 include, but are not limited to, a health care provider 340A (e.g., a dermatologist, a dermatology nurse, a dermatology medical assistant, etc.), a patient 340B, a subject matter expert 340C (e.g., a board-certified dermatologist or other examiner), etc.

In one embodiment, an electronic device 310 comprises one or more sensor units 314 integrated in or coupled to the electronic device 310, such as, but not limited to, a camera, a microphone, a GPS, a motion sensor, a temperature sensor, etc.

In one embodiment, an electronic device 310 comprises a communications unit (i.e., network communications unit) 315 configured to exchange data with a remote computing environment 360 and/or a cosmetic dermatology equipment 330, over a communications network/connection 350 (e.g., a wireless connection such as a Bluetooth® connection, a Wi-Fi connection, or a cellular data connection; a wired connection; or a combination of both a wireless connection and a wired connection). The communications unit 315 may comprise any suitable communications circuitry operative to connect to a communications network and to exchange communications operations and media between the electronic device 310 and other devices connected to the same communications network 350. The communications unit 315 may be operative to interface with a communications network using any suitable communications protocol such as, for example, Wi-Fi (e.g., an IEEE 802.11 protocol), Bluetooth®, high frequency systems (e.g., 900 MHz, 2.4 GHz, and 5.6 GHz communication systems), infrared, GSM, GSM plus EDGE, CDMA, quadband, and other cellular protocols, VOIP, TCP-IP, or any other suitable protocol.

In one embodiment, the remote computing environment 360 includes resources, such as one or more servers 361 and one or more storage units 362. One or more applications that provide higher-level services may execute/operate on the remote computing environment 360 utilizing the resources of the remote computing environment 360. In one embodiment, the one or more applications on the remote computing environment 360 include a server-side cosmetic dermatology system 370. The cosmetic dermatology system 370 is an AI cloud platform for cosmetic dermatology that offers multiple features/functionalities. As described in detail later herein, the cosmetic dermatology system 370 is configured to exchange data with an electronic device 310 via a client-side cosmetic dermatology application 320 on the electronic device 310.

In one embodiment, the remote computing environment 360 provides an online platform for hosting one or more online services (e.g., the server-side cosmetic dermatology system 370, etc.) and/or distributing one or more applications, application updates, and/or AI machine learning models. For example, a client-side cosmetic dermatology application 320 may be loaded onto or downloaded to an electronic device 310 from the remote computing environment 360 that maintains and distributes updates for the application 320, including AI machine learning models. In one embodiment, a client-side cosmetic dermatology application 320 may be downloaded to an electronic device 310 from an application marketplace (e.g., App Store®, Play Store®, etc.) executing/operating on a different remote cloud computing environment.

In one embodiment, the remote computing environment 360 may comprise a cloud computing environment providing shared pools of configurable computing system resources and higher-level services (e.g., data analytics). A client-side cosmetic dermatology application 320 on an electronic device 310 is configured to interface and exchange data with the server-side cosmetic dermatology system 370 in the cloud. In another embodiment, the remote computing environment 360 may comprise an edge computing environment providing more safe, scalable, and reliable data processing and computation.

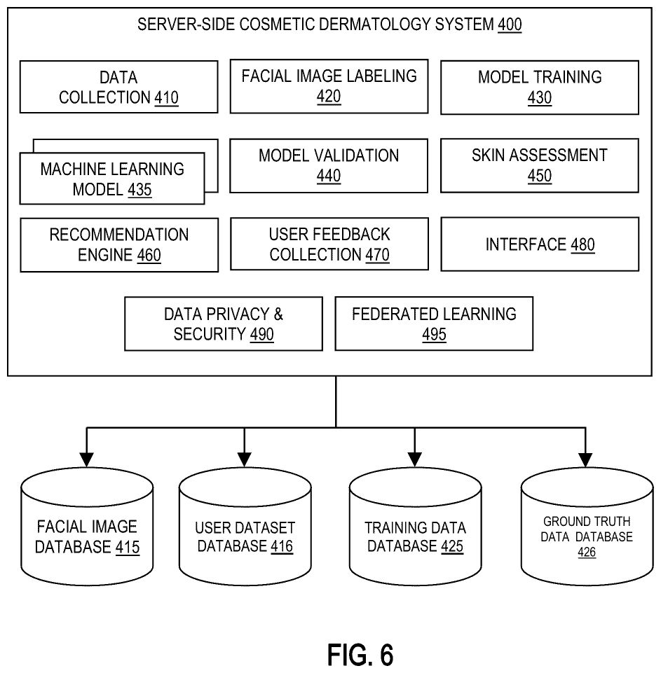

FIG. 6 illustrates an example server-side cosmetic dermatology system 400, in one or more embodiments. The system 400 is an AI-powered cloud platform that offers multiple features/functionalities for cosmetic dermatology applications which users 340 can access via a client-side cosmetic dermatology application 320. In one embodiment, the system 400 is deployed at a remote computing environment 360 (FIG. 5). For example, the system 400 is integrated into, or implemented as part of, the server-side cosmetic dermatology system 370 in FIG. 5.

In one embodiment, the server-side cosmetic dermatology system 400 comprises a data collection unit 410 configured to collect facial images (i.e., photos) from one or more electronic devices 310 (FIG. 5) and/or one or more cosmetic dermatology equipment 330 (FIG. 5). In one embodiment, each facial image collected is maintained on at least one facial image database 415 (e.g., implemented on at least one storage unit 312 (FIG. 5)).

In one embodiment, a cosmetic dermatology equipment 330, such as a patient photography device or clinical/medical imaging device (e.g., VISIA® skin analysis), includes a camera for capturing a facial image of a patient 340B. The captured facial image is uploaded to the system 400 via a client-side cosmetic dermatology application 320 on an electronic device 310 that the cosmetic dermatology equipment 330 is integrated in or coupled to.

In one embodiment, the data collection unit 410 is configured to: (1) provide a questionnaire (i.e., survey) to a client-side cosmetic dermatology application 320 on an electronic device 310/cosmetic dermatology equipment 330 for presentation to a health care provider 340A and/or a patient 340B, (2) collect one or more user responses (e.g., input by a provider 340A or a patient 340B via an I/O unit 313 (FIG. 5)) in response to the questionnaire from the device 310/equipment 330, and (3) generate a user dataset for the patient 340B based on the one or more user responses. A questionnaire includes one or more questions relating to cosmetic dermatology (e.g., questions about skin care routine of a patient 340B, etc.).

In one embodiment, each user dataset generated for each patient 340B is maintained on at least one user dataset database 416 (e.g., implemented on at least one storage unit 362 (FIG. 5)). A user dataset for a patient 340B may include one or more facial images of the patient 340B (e.g., collected via the data collection unit 410). In one embodiment, a user dataset for a patient 340B is formatted/integrated into an electronic medical record for the patient 340B.

In one embodiment, the server-side cosmetic dermatology system 400 comprises a facial image labeling unit 420 configured to: (1) obtain one or more facial images (e.g., from the facial image database 415 and/or an external data source (e.g., a dataset maintained by a third party)), (2) provide the one or more facial images to a client-side cosmetic dermatology application 320 on an electronic device 310 for presentation to a subject matter expert 340C, (3) receive one or more user-provided labels (e.g., input by a subject matter expert 340C via an I/O unit 313 (FIG. 5)) for the one or more facial images from the cosmetic dermatology application 320, and (4) annotate/label the one or more facial images in accordance with the one or more user-provided labels. In one embodiment, some of the resulting annotated/labeled facial images are used as training data for training one or more machine learning models 435. In one embodiment, each annotated/labeled facial image is maintained on at least one training data database 425 (e.g., implemented on at least one storage unit 312 (FIG. 5)). In one embodiment, some of the resulting annotated/labeled facial images are validated (e.g., via a user feedback collection unit 470) and used as ground truth data for validating one or more trained machine learning models 435. In one embodiment, each validated annotated/labeled facial image is maintained on at least one ground truth data database 426 (e.g., implemented on at least one storage unit 312 (FIG. 5)).

In one embodiment, the server-side cosmetic dermatology system 400 comprises a model training unit 430 configured to: (1) obtain training data comprising annotated/labeled facial images (e.g., from the training data database 425), and (2) train, using the training data, one or more machine learning models 435. In one embodiment, the resulting one or more trained machine learning models 435 are configured to: (1) receive, as an input, a facial image of a patient, and (2) based on the facial image, predict one or more skin characteristics of the patient, recommend one or more laser and cosmetic injection treatment plans for the patient, and/or recommend one or more cosmetic dermatology products (e.g., acne products) for the patient.

Examples of a machine learning model 435 include, but are not limited to, a convolutional neural network (CNN), a generative adversarial network (GAN), a recurrent neural network (RNN), a large language model (LLM), etc.

In one embodiment, the server-side cosmetic dermatology system 400 comprises a model validation unit 440 configured to: (1) obtain ground truth data comprising validated annotated/labeled facial images (e.g., from the ground truth data database 426), and (2) validate, using the ground training data, one or more trained machine learning models 435 (e.g., from the model training unit 430). As described in detail later herein, each resulting validated and trained machine learning model 435 is deployed by the system 400 for use in one or more dermatology clinical applications.

In one embodiment, the server-side cosmetic dermatology system 400 comprises a skin assessment unit 450 configured to: (1) receive a facial image of a patient 340B uploaded from an electronic device 310/cosmetic dermatology equipment 330 via a client-side cosmetic dermatology application 320 on the device 310/equipment 330, (2) analyze, utilizing one or more validated and trained machine learning models 435, the facial image to predict one or more skin characteristics of the patient 340B, and (3) provide a skin assessment of the patient 340B to the cosmetic dermatology application 320 on the device 310/equipment 330 for presentation to a health care provider 340A and/or the patient 340B, wherein skin assessment includes the one or more predicted skin characteristics. The skin assessment unit 450 implements predictive analytics/modeling utilizing artificial intelligence (i.e., one or more machine learning models 435) to predict skin characteristics.

Examples of skin characteristics include, but are not limited to, one or more acne severity scores indicative of an amount/degree/level of acne severity, an Investigator Global Assessment (IGA) scale, an amount/degree/level of redness (e.g., a Kesty redness scale), a scale proposed in non-patent literature “Development and Initial Validation of a Multidimensional Acne Global Grading System Integrating Primary Lesions and Secondary Changes” by Bernardis et al., JAMA Dermatol, 2020 (“Bernardis scale”), a skin type (e.g., Fitzpatrick skin type), an amount/degree/level of wrinkle severity (e.g., Glogau aging scale and/or Fitzpatrick wrinkle severity scale), an amount/degree/level of pigmentation (e.g., Kesty hyperpigmentation scale), age, skin condition(s) or disease(s), etc.

In one embodiment, the skin assessment unit 450 determines an overall amount/degree/level of acne severity experienced by a patient 340B by: (1) determining one or more acne severity scores for one or more facial regions of the patient 340B, and (2) determine the overall amount/degree/level of acne severity based on the one or more acne severity scores. In one embodiment, an acne severity score is based on at least one of the following skin characteristics: an IGA scale, a Kesty redness scales, a Bernardis scale, etc.

In one embodiment, the server-side cosmetic dermatology system 400 comprises a recommendation engine 460 implementing an enhanced AI-driven algorithm to suggest or recommend one or more laser and cosmetic injection treatment plans and/or one or more cosmetic dermatology products for a patient 340B based on a skin assessment of the patient 340B (e.g., from the skin assessment unit 450).

Examples of cosmetic dermatology products include, but are not limited to, oral or topical acne care products and treatments, etc. A cosmetic dermatology product may be an over-the-counter product or a prescription medication.

For example, in one embodiment, if a skin assessment (e.g., from the skin assessment unit 450) of a patient 340B includes one or more acne severity scores that exceed a pre-determined threshold, the recommendation engine 460 recommends one or more acne care products and treatments for treating the patient's 340B acne.

In one embodiment, a user dataset/electronic medical record and a medical chart for a patient 340B is updated to include a skin assessment (e.g., from the skin assessment unit 450) of the patient 340B, one or more recommended laser and cosmetic injection treatment plans (e.g., from the recommendation engine 460) for the patient 340B, and/or one or more recommended cosmetic dermatology products (e.g., from the recommendation engine 460) for the patient 340B.

In one embodiment, the AI-driven algorithm of the recommendation engine 460 is configured to: (1) identify, utilizing one or more validated and trained machine learning models 435, intricate patterns and correlations within one or more user datasets (e.g., from the user dataset database 416) for one or more patients 340B, and (2) generate, based on the data identified, curated/personalized recommendations (e.g., recommended laser and cosmetic injection treatment plans, recommended cosmetic dermatology products) for the one or more patients 340B. The recommendation engine 460 provides patients 340B with AI-powered personalized recommendations based on user datasets.

In one embodiment, a recommended laser and cosmetic injection treatment plan for a patient 340B includes one or more recommended settings for a cosmetic dermatology equipment 330 (e.g., laser settings for a therapeutic laser). For example, the treatment plan may suggest one or more lasers that can help the patient 340B and one or more recommended settings for the lasers. Lasers suggested by the recommendation engine 460 may vary for patients 340B based on skin types of the patients 340B (e.g., some lasers are not safe for certain skin types).

In one embodiment, a recommended laser and cosmetic injection treatment plan for a patient 340B identifies one or more injectables that can help the patient 340B, where on the patient 340B to inject the injectables, and how much (e.g., dosages) of the injectables to inject.

In one embodiment, a recommended laser and cosmetic injection treatment plan for a patient 340B identifies one or more acne care products or treatments.

In one embodiment, a recommended laser and cosmetic injection treatment plan for a patient 340B identifies one or more injectables that can help the patient 340B, where on the patient 340B to inject the injectables, and how much (e.g., dosages) of the injectables to inject.

In one embodiment, the recommendation engine 460 is configured to filter and/or rank recommendations for a patient 340B by applying one or more filters and/or ranking criteria to the recommendations, wherein the resulting filtered and/or ranked recommendations is presented (e.g., via a client-side cosmetic dermatology application 320) to the patient 340B and/or health care provider 340A associated with the patient 340B. The one or more filters and/or ranking criteria may be based on location (e.g., availability of treatment plans or products in and around the location), or specific user preferences.

By leveraging one or more machine learning models 435, the system 400 enhances the accuracy and relevance of predictions (e.g., skin assessments) and recommendations (e.g., recommended laser and cosmetic injection treatment plans, recommended cosmetic dermatology products).

In one embodiment, the server-side cosmetic dermatology system 400 comprises a user feedback collection unit 470 configured to collect user feedback (e.g., input via an I/O unit 313 (FIG. 5)) from a user 340. For example, in one embodiment, the user feedback collection unit 470 collects user feedback regarding a skin assessment (e.g., provided by the skin assessment unit 450) of a patient 340B from a health care provide 340A, the patient 340B itself, and/or a subject matter expert 340C. As another example, in one embodiment, the user feedback collection unit 470 collects user feedback regarding a recommendation (e.g., provided by the recommendation engine 460) for a patient 340B from a health care provider 340A, the patient 340B itself, and/or a subject matter expert 340C. As yet another example, in one embodiment, the user feedback collection unit 470 collects user feedback regarding annotated/labeled facial images from a subject matter expert 340C. The user feedback collection unit 470 enables users 340 to provide detailed and explicit input on the accuracy, relevance, and quality of annotated/labeled facial images, skin assessments, and recommendations provided by the system 400.

In one embodiment, user feedback (e.g., collected via the user feedback collection unit 470) is forwarded to one or more components of the server-side cosmetic dermatology system 400, such as the facial image labeling unit 420, the skin assessment unit 450 and the recommendation engine 460, creating a feedback loop and a personalized feedback mechanism in which these components incorporate/integrate the user feedback to continuously improve accuracy, relevance, and quality of ground truth data and future predictions and recommendations. For example, user feedback from a patient 340B may include a rating/review for a particular recommended cosmetic dermatology product for the patient 340B. As another example, user feedback from a health care provider 340B may indicate that a particular recommended laser and cosmetic injection treatment plan for a patient 340B is successful (e.g., improves the patient's 340B skin) or not successful (e.g., no change or worsens the patient's 340B skin). As yet another example, user feedback from a subject matter expert 340C may indicate that a particular skin assessment of a patient 340B or a particular annotated/labeled facial image is accurate or inaccurate. Such user-provided inputs are used to refine and/or validate one or more machine learning models 435 utilized by the system 400. The system 400 is able to determine, based on user feedback, historical/past success metrics/rates that are indicative of whether past skin assessments were accurate and whether past recommendations were successful. The system 400 leverages user feedback to continuously fine-tune/refine/update and validate the machine learning models 435 and in turn improve the overall user experience of users 340.

The server-side cosmetic dermatology system 400 integrates, via the feedback loop, a novel adaptive and continuous learning framework that continuously adapts and refines one or more machine learning models 435 utilized by the system 400 based on user feedback and historical/past success metrics/rates. This iterative process ensures that the system 400 remains attuned to an ever-evolving cosmetic dermatology industry to be able to provide users 340 with optimal results, i.e., accurate and up-to-date predictions and recommendations. Therefore, the system 400, via the machine learning models 435, implements advanced data analytics that uses predictive analytics to consider historical data (e.g., historical/past success metrics/rates) and predict future trend projections (e.g., emerging trends in the cosmetic dermatology industry).

For example, if a relatively new cosmetic dermatology product recommended to a first set of patients 340B receives good ratings/reviews from the first set of patients 340B, the server-side cosmetic dermatology system 400 is more likely to recommend the same product to another set of patients 340B with similar skin characteristics as the first set of patients 340B.

In one embodiment, a user dataset/electronic medical record and a medical chart for a patient 340B is updated to include user feedback (e.g., collected by the user feedback collection unit 470) from the patient 340B and/or a health care provider 340A associated with the patient 340B.

In one embodiment, the ground truth data database 426 is updated to include: (1) each facial image of each patient 340B with an accurate skin assessment (based on user feedback), and (2) each annotated/labeled facial image that is validated as accurate (based on user feedback).

In one embodiment, the server-side cosmetic dermatology system 400 comprises an interface unit 480 configured to generate one or more GUIs for display to a user 340 (e.g., via a client-side cosmetic dermatology application 320, a web browser, or an I/O unit 113, such as a GUI). For example, in one embodiment, the interface unit 480 receives a skin assessment for a patient 340B (e.g., from the skin assessment unit 450), and generates one or more GUIs including the skin assessment for presentation on an electronic device 310/cosmetic dermatology equipment 330. As another example, in one embodiment, the interface unit 480 receives one or more recommendations for a patient 340B (e.g., from the recommendation engine 460), and generates one or more GUIs including the one or more recommendations for presentation on an electronic device 310/cosmetic dermatology equipment 330. The interface unit 480 may also generate one or more GUIs including a questionnaire, a prompt for user feedback, a prompt to validate an annotated/labeled facial image, etc.

In one embodiment, the interface unit 480 is configured to trigger automatic configuration of a cosmetic intervention procedure (e.g., laser treatment using a therapeutic laser) involving a patient 340B in accordance with a recommended treatment plan (e.g., from the recommendation engine 460) for the patient 340B. For example, in one embodiment, the interface unit 480 triggers automatic configuration of a cosmetic dermatology equipment 330 (e.g., a therapeutic laser) based on one or more recommended settings included in a recommended treatment plan. In one embodiment, the interface unit 480 triggers the automatic configuration via a client-side cosmetic dermatology application 320 on an electronic device 310 that the equipment 330 is integrated in or coupled to.

The interface unit 480 enables automatically configuring/programming/setting cosmetic dermatology equipment 330 in accordance with a recommended treatment plan for a patient 340B. A health care provider 340A can then treat the patient 340B using the equipment 330, without having to rely on themselves or another person to read recommended settings included in the treatment plan and manually program the equipment 330 based on the recommended settings (i.e., manually input the recommended settings into the equipment 330). The ability to automatically configure/program/set the equipment 330 with recommended settings removes human error. For example, if a recommended treatment plan recommends a laser and includes laser settings for the laser, the system 400 can invoke (e.g., via the interface unit 480) automatic configuration of the laser based on the laser settings.

In one embodiment, the interface unit 480 is configured to generate a virtual patient portal dashboard that one or more patients 340B can access (e.g., via a client-side cosmetic dermatology application 320 or a web browser). For each patient 340B, the dashboard includes one or more GUIs including patient information for the patient 340B that is obtained from a user dataset of the patient 340B (e.g., from the user dataset database 416). Patient information includes, but is not limited to, one or more facial images, one or more skin assessments, one or more recommendations, etc. Patients 340B can securely access their electronic medical records via the dashboard. In one embodiment, access to the dashboard is password-protected for privacy—each patient 340B will have to create an account before they can utilize the dashboard, upload a facial image for skin assessment, etc.

In one embodiment, the server-side cosmetic dermatology system 400 comprises a data privacy and security unit 490 that implements one or more user data protection measures (or one or more data privacy and security protocols) to ensure the confidentiality and protection of user datasets for patients 340B, as well as comply with industry standards and regulations (e.g., Health Insurance Portability and Accountability Act (HIPAA)). For example, in one embodiment, the 490 utilizes one or more advanced/robust data encryption protocols, and/or one or more secure data transmission channels to ensure user data privacy and confidentiality. As another example, in one embodiment, the unit 490 implements one or more user permission protocols in which the unit 490 obtains and manages user consent from each patient 340B for collection and analysis of user data (e.g., facial image, user responses) relating to the patient 340B. As yet another example, in one embodiment, the unit 490 safeguards private/sensitive user information utilizing secure data storage and one or more advanced/robust authentication protocols to protect user data integrity.

In one embodiment, a client-side cosmetic dermatology application 320 includes one or more components (e.g., similar to one or more components of the system 400) that provide features/functionalities as described herein above.

In one embodiment, one or more validated and trained machine learning models 435 are downloaded to an electronic device 310/cosmetic dermatology equipment 330 for use by a client-side cosmetic dermatology application 320 on the device 310/equipment 330. The application 320 can provide skin assessments and recommendations without having to exchange data with the system 400. This mitigates the risk of compromising data security or privacy as the device 310/equipment 330 need not share private data-such as user responses, facial images—with the system 400.

In one embodiment, the server-side cosmetic dermatology system 400 comprises an optional federated learning unit 495 for implementing federated learning. Specifically, the federated learning unit 495 is configured to coordinate the collaborative training of one or more global AI machine learning models across one or more electronic devices 310 and/or one or more cosmetic dermatology equipment 330 in similar contexts (e.g., similar geographical locations, similar skin characteristics, etc.). For example, an initial global AI machine learning model is deployed to a group of electronic devices 310 and/or cosmetic dermatology equipment 330 in a similar context (e.g., same geographical location). Each device 310/equipment 330 of the group locally trains (e.g., via a client-side cosmetic dermatology application 320 on the device 310/equipment 330) the initial global AI machine learning model based on user feedback, resulting in a local AI machine learning model that is then shared with the federated learning unit 495. The federated learning unit 495 fine-tunes/refines/updates the global AI machine learning model based on each local AI machine learning model it receives from each device 310/equipment 330 of the group. This mitigates the risk of compromising data security or privacy as the devices 310/equipment 330 never share private data—user feedback—with the federated learning unit 495.

In one embodiment, a user 340 may require an active subscription in order to access features/functionalities as described herein above.

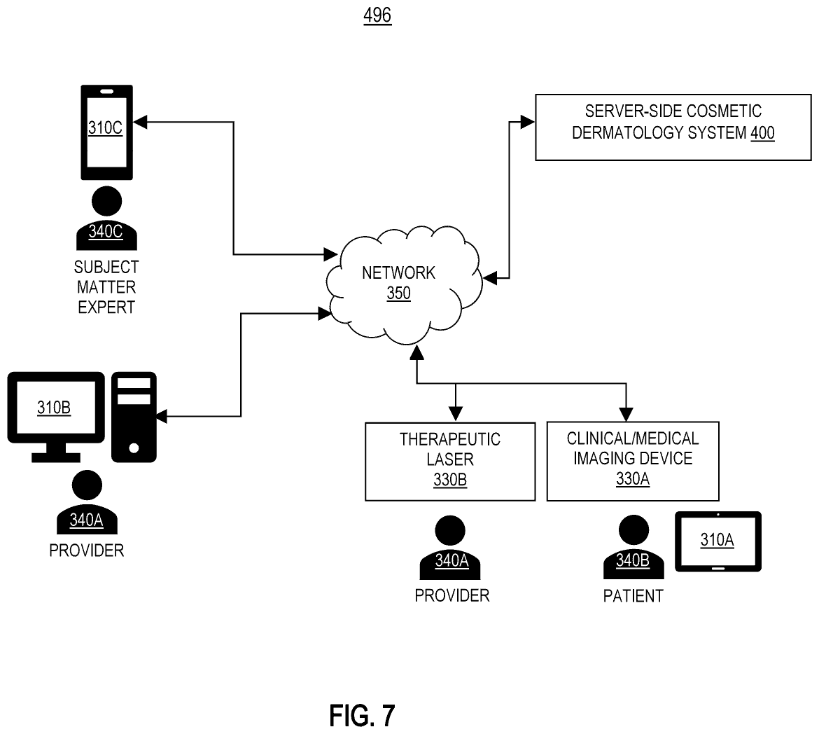

FIG. 7 illustrates an example ecosystem 496, in one or more embodiments. As shown in FIG. 7, one or more electronic devices 310 and/or one or more cosmetic dermatology equipment 330 may be connected to the server-side cosmetic dermatology system 400 to create the ecosystem 496. For example, a facial image (i.e., photo) of a patient 340B is captured and uploaded to the system 400 using a first connected device 310/equipment 330 (e.g., a clinical/medical imaging device 330A). As another example, the patient 340B provides user responses or user feedback to the system 400 using a second connected device 310/equipment 330 (e.g., a tablet 310A). As another example, a health care provider 340A (e.g., a dermatologist) reviews a skin assessment/recommendation/electronic medical record from the system 400 for the patient 340B using a third connected device 310/equipment 330 (e.g., a desktop computer 310B). As another example, a fourth connected device 310/equipment 330 (e.g., a therapeutic laser 330B) is automatically configured/programmed/set by the system 400; another healthcare provider 340A (e.g., a dermatology medical assistant) then uses the fourth connected device 310/equipment 330 for treatment of the patient 340B without having to manually configure the device 310/equipment 330. The first, second, third, and fourth connected devices 310/equipment 330 may be the same device 310/equipment 330 or different devices 310/equipment 330. As another example, a subject matter expert 340C either provides user feedback or annotates/validates the facial image using a fifth connected device 310/equipment 330 (e.g., a smart phone 310C), such that the user feedback or the facial image is used by the system 400 to train/validate/refine one or more machine learning models.

As demand for cosmetic dermatology procedures increase year after year, cosmetic dermatology professionals trained and certified to perform cosmetic interventions struggle to keep up with the demand. Embodiments of the inventions solve this problem by decreasing training requirements for operating lasers and providing treatment plans for lasers, injectables, and acne, thereby saving time and costs involved with training health care providers to operate lasers, etc.

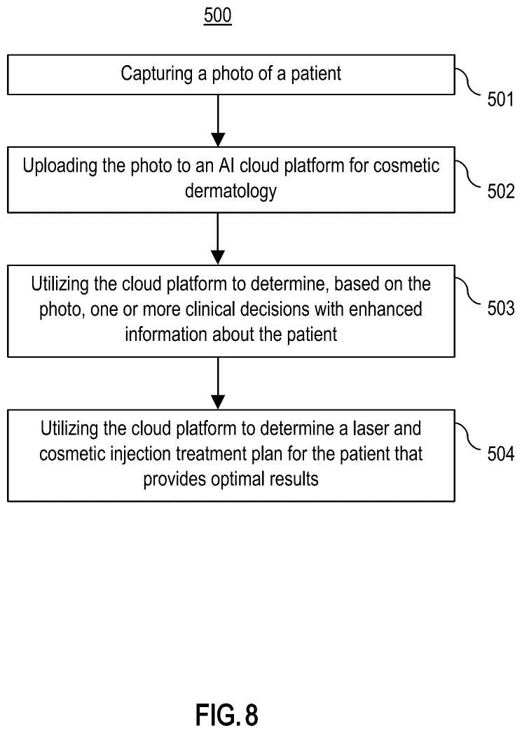

FIG. 8 is a flowchart of an example process 500 for utilizing an AI cloud platform for cosmetic dermatology (e.g., the server-side cosmetic dermatology system 400), in one or more embodiments. Process block 501 includes capturing a photo of a patient (e.g., using an electronic device 310 or a cosmetic dermatology equipment 330). Process block 502 includes uploading the photo to the AI cloud platform. Process block 503 includes utilizing the cloud platform to determine (e.g., via the skin assessment unit 450), based on the photo, one or more clinical decisions with enhanced information about the patient (e.g., skin assessment including skin characteristics of the patient). Process block 504 includes utilizing the cloud application to determine (e.g., via the recommendation engine 460) a laser and cosmetic injection treatment plan for the patient that provides optimal results (e.g., one or more recommended laser and cosmetic injection treatment plans for the patient, one or more recommended cosmetic dermatology products for the patient).

In one embodiment, process blocks 501-504 may be performed utilizing one or more components of the system 400, the system 100, the system 130, the system 140, the system 160, and/or the application 320.

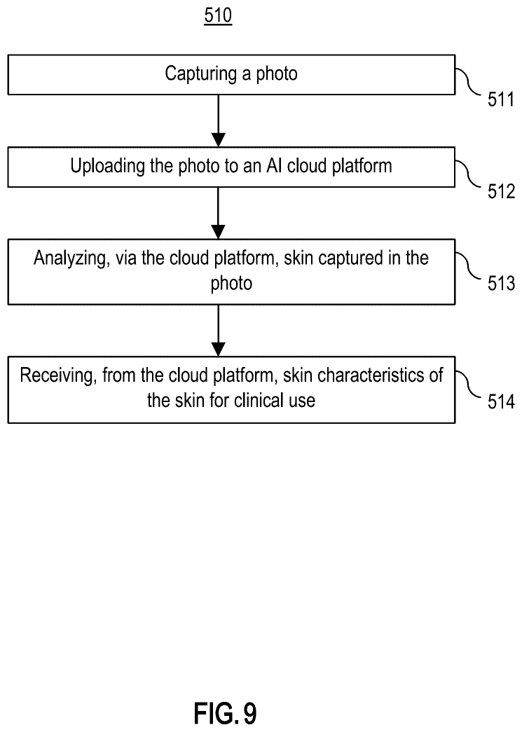

FIG. 9 is a flowchart of another example process 510 for utilizing an AI cloud platform for cosmetic dermatology (e.g., the server-side cosmetic dermatology system 400), in one or more embodiments. Process block 511 includes capturing a photo (e.g., using an electronic device 310 or a cosmetic dermatology equipment 330). Process block 512 includes uploading the photo to the AI cloud platform. Process block 513 includes analyzing (e.g., via the skin assessment unit 450), via the cloud platform, skin captured in the photo. Process block 514 includes receiving, from the cloud platform, skin characteristics of the skin for clinical use (e.g., a skin assessment including skin characteristics from the skin assessment unit 450).

In one embodiment, process blocks 511-514 may be performed utilizing one or more components of the system 400, the system 100, the system 130, the system 140, the system 160, and/or the application 320.

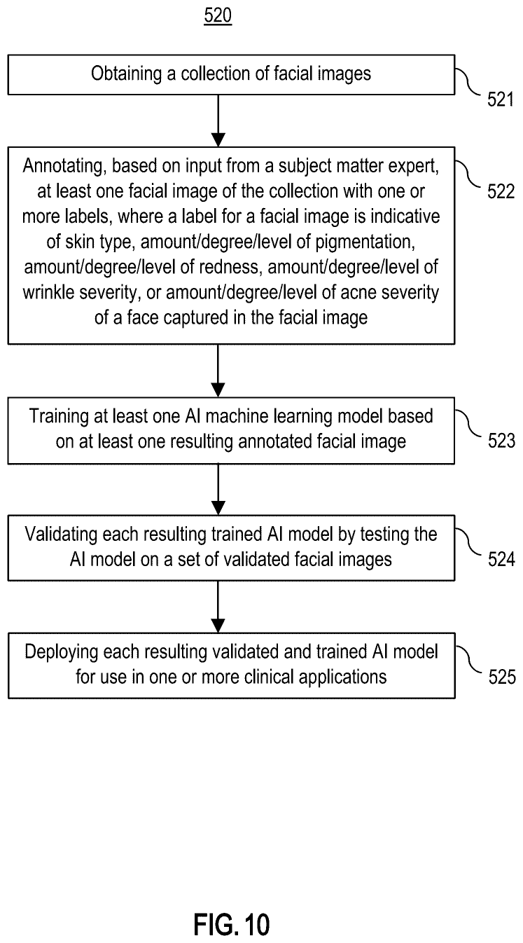

FIG. 10 is a flowchart of an example process 520 for training machine learning models, in one or more embodiments. Process block 521 includes obtaining (e.g., via the data collection unit 410) a collection of facial images (e.g., from one or more electronic device 310 and/or cosmetic dermatology equipment 330). Process block 522 includes annotating (e.g., via the facial image labeling unit 420), based on input from a subject matter expert (e.g., subject matter expert 340C), at least one facial image of the collection with one or more labels, where a label for a facial image is indicative of skin type, amount/degree/level of pigmentation, amount/degree/level of redness, amount/degree/level of wrinkle severity, or amount/degree/level of acne severity of a face captured in the facial image. Process block 523 includes training (e.g., via the model training unit 430) at least one AI machine learning model (e.g., machine learning models 435) based on at least one resulting annotated facial image. Process block 524 includes validating (e.g., via the model validation unit 440) each resulting trained AI model by testing the AI model on a set of validated facial images. Process block 525 includes deploying each resulting validated and trained AI model (e.g., machine learning models 435) for use in one or more clinical applications validating (e.g., by the skin assessment unit 450, the recommendation engine 460, downloaded to an electronic device 310 or cosmetic dermatology equipment 330) for use by a client-side cosmetic dermatology application 320.

In one embodiment, process blocks 521-525 may be performed utilizing one or more components of the system 400, the system 100, the system 130, the system 140, the system 160, and/or the application 320.

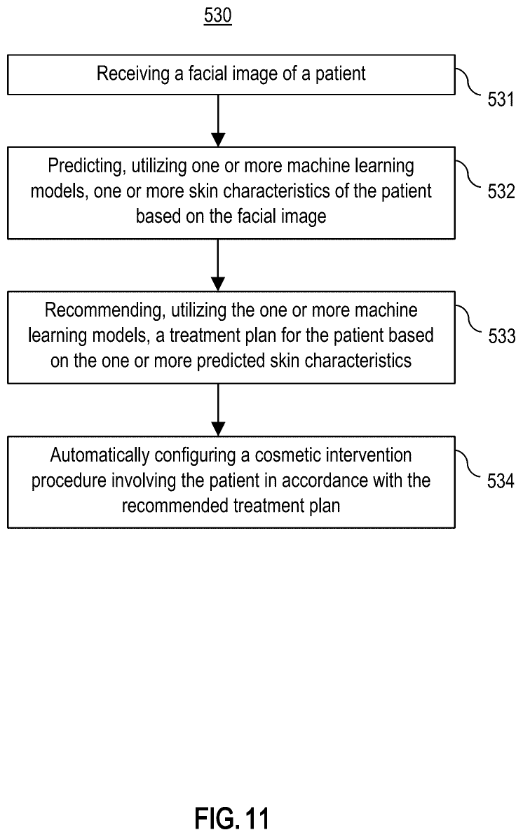

FIG. 11 is a flowchart of an example process 530 implemented by an AI cloud platform (e.g., the server-side cosmetic dermatology system 400), in one or more embodiments. Process block 531 includes receiving (e.g., via the data collection unit 410) a facial image of a patient (e.g., from one or more electronic device 310 and/or cosmetic dermatology equipment 330). Process block 532 includes predicting (e.g., via the skin assessment unit 450), utilizing one or more machine learning models (e.g., the machine learning models 435), one or more skin characteristics of the patient based on the facial image. Process block 533 includes recommending (e.g., via the recommendation engine 460), utilizing the one or more machine learning models, a treatment plan for the patient based on the one or more predicted skin characteristics. Process block 534 includes automatically configuring (e.g., via the interface unit 480) a cosmetic intervention procedure involving the patient in accordance with the recommended treatment plan.

In one embodiment, process blocks 531-534 may be performed utilizing one or more components of the system 400, the system 100, the system 130, the system 140, the system 160, and/or the application 320.

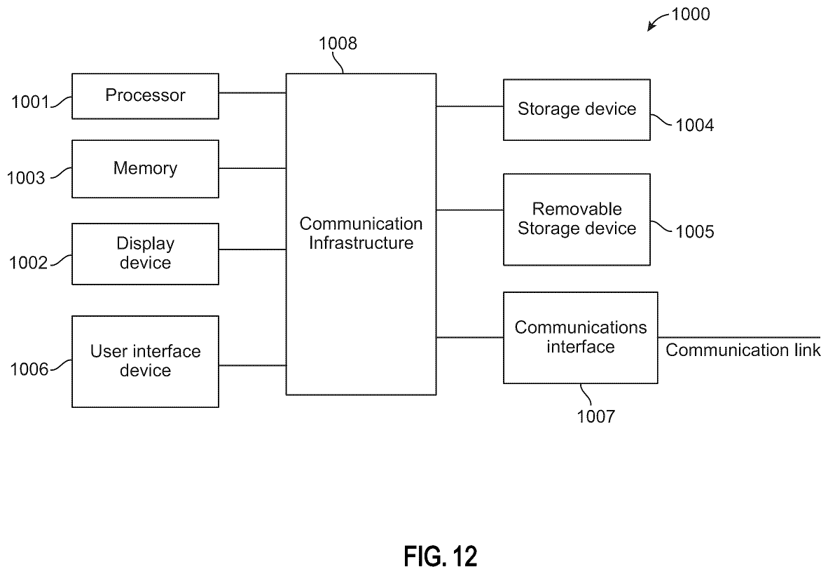

FIG. 12 is a high-level block diagram showing an information processing system comprising a computer system 1000 useful for implementing the disclosed embodiments. The computer system 1000 includes one or more processors 1001, and can further include an electronic display device 1002 (for displaying video, graphics, text, and other data), a main memory 1003 (e.g., random access memory (RAM)), storage device 1004 (e.g., hard disk drive), removable storage device 1005 (e.g., removable storage drive, removable memory module, a magnetic tape drive, optical disk drive, computer readable medium having stored therein computer software and/or data), user interface device 1006 (e.g., keyboard, touch screen, keypad, pointing device), and a communication interface 1007 (e.g., modem, a network interface (such as an Ethernet card), a communications port, or a PCMCIA slot and card). The main memory 1003 may store instructions that when executed by the one or more processors 1001 cause the one or more processors 1001 to perform one or more process blocks of the process 950.

The communication interface 1007 allows software and data to be transferred between the computer system and external devices. The system 1000 further includes a communications infrastructure 1008 (e.g., a communications bus, cross-over bar, or network) to which the aforementioned devices/modules 1001 through 1007 are connected.

Information transferred via communications interface 1007 may be in the form of signals such as electronic, electromagnetic, optical, or other signals capable of being received by communications interface 1007, via a communication link that carries signals and may be implemented using wire or cable, fiber optics, a phone line, a cellular phone link, a radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process. In one embodiment, processing instructions for one or more process blocks of methods/processes 170-190 (FIGS. 2A-2C), 260 (FIG. 2D), and 500-530 (FIGS. 8-11) may be stored as program instructions on the memory 1003, storage device 1004 and the removable storage device 1005 for execution by the processor 1001.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A method comprising:
receiving a facial image of a patient;
predicting, utilizing one or more machine learning models, one or more skin characteristics of the patient based on the facial image;
recommending, utilizing the one or more machine learning models, a treatment plan for the patient based on the one or more predicted skin characteristics; and
automatically configuring a cosmetic intervention procedure involving the patient in accordance with the recommended treatment plan.

2. The method of claim 1, further comprising:
receiving one or more user responses to a questionnaire from the patient or a health care provider associated with the patient, wherein the predicting is further based on the one or more user responses.

3. The method of claim 1, wherein the one or more predicted skin characteristics include at least one of: skin type, pigmentation scale, redness scale, wrinkle severity score, acne severity score, age, skin disease, or skin condition.

4. The method of claim 1, wherein the recommended treatment plan recommends one or more lasers and one or more laser settings for the one or more lasers.

5. The method of claim 4, wherein the automatically configuring comprises:
automatically setting the one or more lasers based on the one or more laser settings.

6. The method of claim 1, wherein the recommended treatment plan recommends one or more injectables, one or more locations on the patient to inject the one or more injectables, and one or more dosages of the one or more injectables to inject at the one or more locations.

7. The method of claim 1, wherein the recommended treatment plan recommends one or more acne care products or treatments.

8. The method of claim 1, further comprising:
collecting user feedback from the patient, a health care provider associated with the patient, or a subject matter expert;
updating the one or more machine learning models based on the user feedback.

9. The method of claim 1, further comprising:
updating an electrical medical record for the patient to include the facial image, the one or more predicted skin characteristics, and the recommended treatment plan.

10. The method of claim 9, further comprising:
providing a virtual patient portal dashboard including patient access to the electrical medical record.

11. A system comprising:
at least one processor; and
a non-transitory processor-readable memory device storing instructions that when executed by the at least one processor causes the at least one processor to perform operations including:
receiving a facial image of a patient;
predicting, utilizing one or more machine learning models, one or more skin characteristics of the patient based on the facial image;
recommending, utilizing the one or more machine learning models, a treatment plan for the patient based on the one or more predicted skin characteristics; and
automatically configuring a cosmetic intervention procedure involving the patient in accordance with the recommended treatment plan.

12. The system of claim 11, wherein the operations further include:
receiving one or more user responses to a questionnaire from the patient or a health care provider associated with the patient, wherein the predicting is further based on the one or more user responses.

13. The system of claim 11, wherein the one or more predicted skin characteristics include at least one of: skin type, pigmentation scale, redness scale, wrinkle severity score, acne severity score, age, skin disease, or skin condition.

14. The system of claim 11, wherein the recommended treatment plan recommends one or more lasers and one or more laser settings for the one or more lasers.

15. The system of claim 14, wherein the automatically configuring comprises:
automatically setting the one or more lasers based on the one or more laser settings.

16. The system of claim 11, wherein the recommended treatment plan recommends one or more injectables, one or more locations on the patient to inject the one or more injectables, and one or more dosages of the one or more injectables to inject at the one or more locations.

17. The system of claim 11, wherein the recommended treatment plan recommends one or more acne care products or treatments.

18. The system of claim 11, wherein the operations further include:
collecting user feedback from the patient, a health care provider associated with the patient, or a subject matter expert; and
updating the one or more machine learning models based on the user feedback.

19. The system of claim 11, wherein the operations further include:
updating an electrical medical record for the patient to include the facial image, the one or more predicted skin characteristics, and the recommended treatment plan; and
providing a virtual patient portal dashboard including patient access to the electrical medical record.

20. A non-transitory processor-readable medium that includes a program that when executed by a processor performs a method comprising:
receiving a facial image of a patient;
predicting, utilizing one or more machine learning models, one or more skin characteristics of the patient based on the facial image;
recommending, utilizing the one or more machine learning models, a treatment plan for the patient based on the one or more predicted skin characteristics; and
automatically configuring a cosmetic intervention procedure involving the patient in accordance with the recommended treatment plan.
